(19) United States
(12) Patent Application Publication
(10) Pub. No.: US 2018/0140200 A1
(43) Pub. Date: May 24, 2018

(54) PROBE ARRAY, ACOUSTIC WAVE UNIT, AND INFORMATION ACQUISITION APPARATUSES USING SAME

(71) Applicant: CANON KABUSHIKI KAISHA, Tokyo (JP)

(72) Inventors: Toshifumi Yoshioka, Hiratsuka-shi (JP); Yoshiki Kino, Yokohama-shi (JP)

(21) Appl. No.: 15/812,957

(22) Filed: Nov. 14, 2017

(30) Foreign Application Priority Data
Nov. 18, 2016 (JP) 2016-225553
Dec. 27, 2016 (JP) 2016-254366
Dec. 27, 2016 (JP) 2016-254367

(51) Int. Cl. A61B 5/00

(52) U.S. Cl. CPC A61B 5/0095; A61B 2562/046

(57) ABSTRACT

A probe array in which probes are arranged on a cup-shaped acoustic wave detector at high density. In the probe array, transducers (acoustic wave conversion elements) configured to be capable of conversion between acoustic waves and an electrical signal are provided on a support member (housing).

### BACKGROUND OF THE INVENTION

### Field of the Invention

The present disclosure relates to a probe array and an acoustic wave unit for detecting acoustic waves, and information acquisition apparatuses using the same.

### Description of the Related Art

There are techniques for using light having a wavelength of approximately 600 nm to 1500 nm, which has excellent transmission characteristics to living tissue, to determine the formation of new blood vessels and the oxygen metabolism of hemoglobin due to growth of tumors from light absorption characteristics of hemoglobin included in the blood and use the determination for tumor diagnosis. Among such techniques is the use of a photoacoustic effect.

The photoacoustic effect refers to a phenomenon in which if a substance is irradiated with pulsed light of the order of nanoseconds, the substance absorbs light energy according to its light absorption characteristics and the substance expands instantaneously to generate elastic waves. The elastic waves are detected by ultrasonic probes to obtain reception signals. The reception signals are mathematically analyzed and processed, whereby absorption characteristics inside the living body can be imaged based on a sound pressure distribution of the elastic waves generated by the photoacoustic effect. Hemoglobin has high near-infrared absorptivity, compared to water, fat, and protein constituting living tissue. The foregoing method is thus suited for measuring new blood vessels and oxygen metabolism. Clinical research for using and applying such a photoacoustic effect to the diagnosis of breast cancer has been advanced.

As a photoacoustic apparatus using the photoacoustic effect, WO 2010/030817 discusses a configuration including a cup-shaped acoustic wave detector on which a plurality of acoustic elements (transducers) is spirally arranged. This apparatus includes a light irradiation unit for irradiating a subject with light. The irradiation unit is provided in a lower part of the cup-shaped acoustic wave detector. With such an apparatus configuration, acoustic waves from the subject are received in multiple directions to improve the resolution of the obtained image.

An apparatus for obtaining a subject image with high accuracy may be configured such that probes including acoustic elements are mounted at high density. The spiral arrangement of acoustic elements as discussed in WO 2010/030817 is a method capable of high density arrangement. According to the method, however, the acoustic elements are unevenly arranged. The probes therefore need to be arranged not to interfere with each other when fixed to the acoustic wave detector.

For stable fixing, if fixing portions are flat surfaces, portions to be fixed are typically shaped to be flat as well. If the probes have flat fixing portions, the portions to be fixed of the acoustic wave detector therefore is desirably cut into flat shapes according to the fixing portions of the probes.

If the outer surface of the acoustic wave detector has a sufficiently large surface area with respect to the total area of the fixing portions of the probes, the cut surfaces do not intersect with each other. On the other hand, if the outer surface of the acoustic wave detector has a surface area close to the total area of the fixing portions of the probes, the cut surfaces can intersect or the ridges constituting the cut surfaces approach each other. In the former case, there is a problem that if fixing places are included in the cut surfaces, the fixing places can be chipped in part. In the latter case, there can occur small ribs or edges which hinder assembly operations. That is, problems with the formation of flat portions to be fixed in association with flat fixing portions have been found.

As described above, in obtaining a subject image with high accuracy, the probes including the acoustic elements need to be arranged at high density. The surface area of the outer surface of the acoustic wave detector thus approaches the total area of the fixing portions of the probes. The problems found by the present inventor then need to be solved, whereas no solution has been known heretofore.

### SUMMARY OF THE INVENTION

The present disclosure is directed to the provision of a probe array in which probes are arranged on a cup-shaped acoustic wave detector at high density and portions to be fixed to which the probes are fixed do not need to be flat surfaces.

According to an aspect of the present disclosure, a probe array includes a cup-shaped support member having a plurality of through holes, and a probe provided in a through hole of the plurality of through holes, the probe including a transducer configured to be capable of conversion between an acoustic wave and an electrical signal, wherein the probe include a fixing portion for fixing the probe to an outer surface of the support member, the fixing portion having a curvature.

According to another aspect of the present disclosure, a probe array includes a cup-shaped support member having a plurality of holes, and a cylindrical probe provided in a hole of the plurality of holes, the cylindrical probe including a transducer configured to be capable of conversion between an acoustic wave and an electrical signal, wherein an axis of rotational symmetry of the cup-shaped support member and normals from points at which a curved surface including an inside surface of the cup-shaped support member intersects with center axes of the cylindrical probes to the axis of rotational symmetry have a plurality of intersections, and wherein a distance between a first intersection among the plurality of intersections and a second intersection adjoining the first intersection on an apex side of the cup-shaped support member is greater than a distance between the first intersection and a third intersection adjoining the first intersection on a side opposite from the apex side.

According to yet another aspect of the present disclosure, an acoustic wave unit includes a housing configured to hold a plurality of probes arranged thereon, each probe of the plurality of probes including an acoustic wave conversion element configured to receive an acoustic wave from a subject and convert information about the acoustic wave into an electrical signal, each probe of the plurality of probes being provided to protrude in part from a surface of the housing on a side opposite from a surface opposed to the subject, wherein each probe of the plurality of probes has a protruding length from the surface on the opposite side that is different from another probe of the plurality of probes.

According to a probe array according to a first exemplary embodiment, if probes are provided at high density on a cup-shaped support member having a plurality of through holes, the support member does not need to be machined into flat shapes since fixing portions for fixing the probes have a curvature. A highly accurate subject image can be obtained by using an information acquisition apparatus including such a probe array on which probes are provided at high density.

Further features of the present disclosure will become apparent from the following description of exemplary embodiments with reference to the attached drawings.

### BRIEF DESCRIPTION OF THE DRAWINGS

FIG. 1A is a sectional view illustrating a configuration of a probe array according to exemplary embodiment 1 of a first exemplary embodiment.

FIG. 1B is an enlarged sectional view of a part of the probe array according to exemplary embodiment 1 of the first exemplary embodiment.

FIG. 1C-1 is a sectional view illustrating a transducer according to exemplary embodiment 1 of the first exemplary embodiment in detail.

FIG. 1C-2 is a perspective view illustrating the transducer according to exemplary embodiment 1 of the first exemplary embodiment in detail.

FIG. 1D-1 is a sectional view when an outer surface of a support member according to exemplary embodiment 1 of the first exemplary embodiment has a curvature smaller than that provided on fixing portions.

FIG. 1D-2 is a sectional view when the outer surface of the support member according to exemplary embodiment 1 of the first exemplary embodiment has a curvature greater than that provided on the fixing portions 110.

FIG. 1E is a sectional view of a transducer having notches in part of the fixing portions, according to exemplary embodiment 1 of the first exemplary embodiment.

FIG. 1F is an enlarged view seen from an outer surface side when probes 103 according to exemplary embodiment 1 of the first exemplary embodiment are arranged on the support member.

FIG. 1G-1 is a diagram for describing a comparative example of the probe according to exemplary embodiment 1 of the first exemplary embodiment.

FIG. 1G-2 is a diagram illustrating a state in which flat surface portions are formed according to the surfaces of the fixing portions to be attached to the support member so that the probes according to exemplary embodiment of the first exemplary embodiment are arranged on the support member.

FIG. 1H-1 illustrates reference example 1 of the probe according to exemplary embodiment 1 of the first exemplary embodiment.

FIG. 1H-2 illustrates reference example 2 of the probe according to exemplary embodiment 1 of the first exemplary embodiment.

FIG. 1I-1 is a diagram in which positioning mechanisms are provided on fixing portions of a probe according to exemplary embodiment 2 of the first exemplary embodiment.

FIG. 1I-2 is a diagram in which the positioning mechanisms are provided on the fixing portions of the probe according to exemplary embodiment 2 of the first exemplary embodiment.

FIG. 1I-3 is a diagram illustrating a hole structure on the side of a housing 104 according to exemplary embodiment 2 of the first exemplary embodiment.

FIG. 1I-4 is a diagram illustrating the hole structure on the side of the housing 104 according to exemplary embodiment 2 of the first exemplary embodiment.

FIG. 1J-1 illustrates an information acquisition apparatus according to exemplary embodiment 3 of the first exemplary embodiment.

FIG. 1J-2 illustrates an information acquisition apparatus according to exemplary embodiment 4 of the first exemplary embodiment.

FIG. 2A is a sectional view illustrating a configuration of an acoustic wave unit 100 according to exemplary embodiment 1 of a second exemplary embodiment.

FIG. 2B is a sectional view illustrating an ultrasonic probe 103 according to exemplary embodiment 1 of the second exemplary embodiment in detail.

FIG. 2C is a diagram illustrating a shape of the ultrasonic probe 103 according to exemplary embodiment 1 of the second exemplary embodiment in a perspective manner.

FIG. 2D is a diagram in which some of ultrasonic probes 103 are displayed on a bottom side of a housing 104 according to exemplary embodiment 1 of the second exemplary embodiment.

FIG. 2E is a detailed view of a state of arrangement of the ultrasonic probes 103 attached onto the outer surface of the housing 104 according to exemplary embodiment 1 of the second exemplary embodiment.

FIG. 2F is an enlarged view of the arrangement of the ultrasonic probes 103 in a bottommost portion of the housing 104 according to exemplary embodiment 1 of the second exemplary embodiment.

FIG. 2G is an enlarged view of the arrangement of the ultrasonic probes 103 in a position farthest from the bottommost portion of the housing 104 according to exemplary embodiment 1 of the second exemplary embodiment.

FIG. 2H is a diagram illustrating that reference points of the first and second ultrasonic probes 103 according to exemplary embodiment 1 of the second exemplary embodiment are projected upon an axis of rotational symmetry of the housing 104, connecting the bottommost portion of the housing 104 to a subject 102.

FIG. 2I is a diagram illustrating that the first, 14th, 22nd, and 35th ultrasonic probes 103 according to exemplary embodiment 1 of the second exemplary embodiment are projected upon an arbitrary plane perpendicular to the axis of rotational symmetry connecting the bottommost portion of the housing 104 to the subject 102.

FIG. 2J is a diagram illustrating an alternative structure of the ultrasonic probe 103 according to exemplary embodiment 1 of the second exemplary embodiment.

FIG. 2K is a diagram illustrating a case in which the structure illustrated in FIG. 2J is used in FIG. 21 according to exemplary embodiment 1 of the second exemplary embodiment.

FIG. 2L is a diagram of a second alternative structure of the ultrasonic probe 103 according to exemplary embodiment 1 of the second exemplary embodiment.

FIG. 2M is a diagram of a relationship between a constant A and a value obtained by dividing “i” by “s−1”, according to exemplary embodiment 2 of the second exemplary embodiment.

FIG. 2N is a representation using specific values (constant A=2, s=20) according to exemplary embodiment 2 of the second exemplary embodiment.

FIG. 20 is a representation using specific values (constant A=5, s=20) according to exemplary embodiment 2 of the second exemplary embodiment.

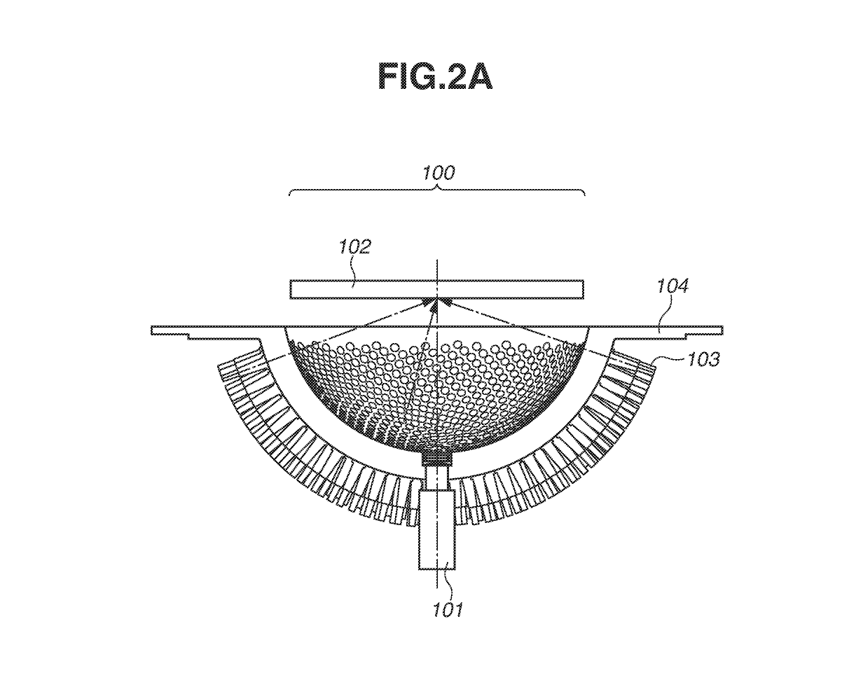

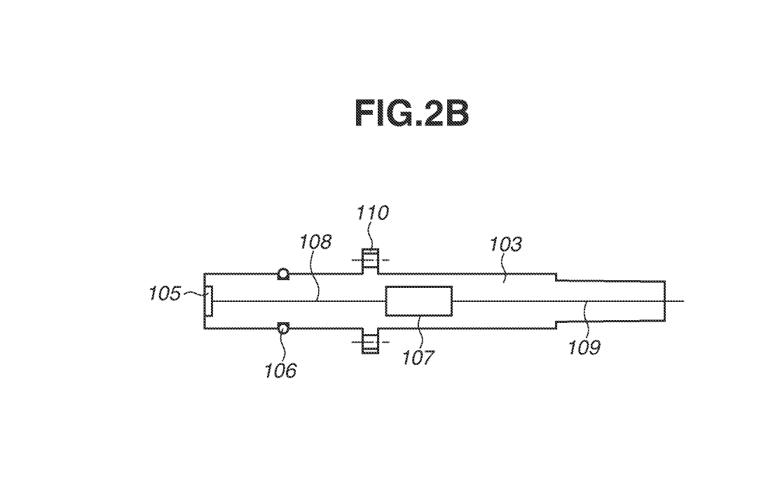

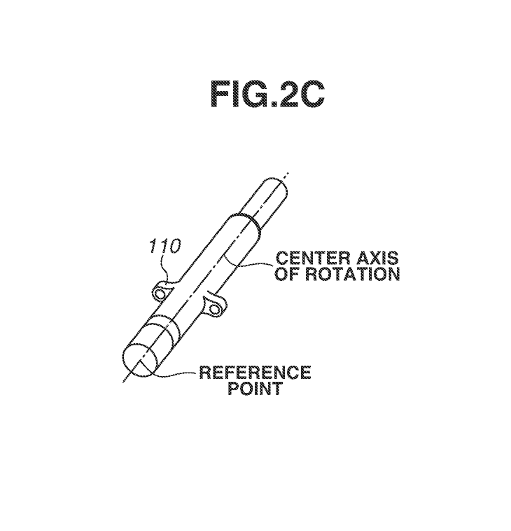

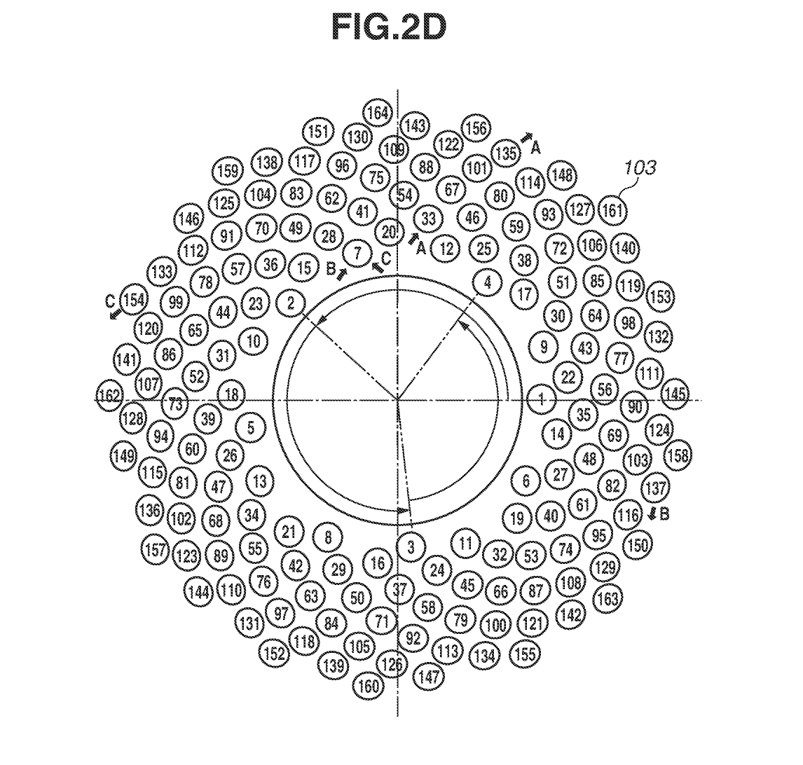

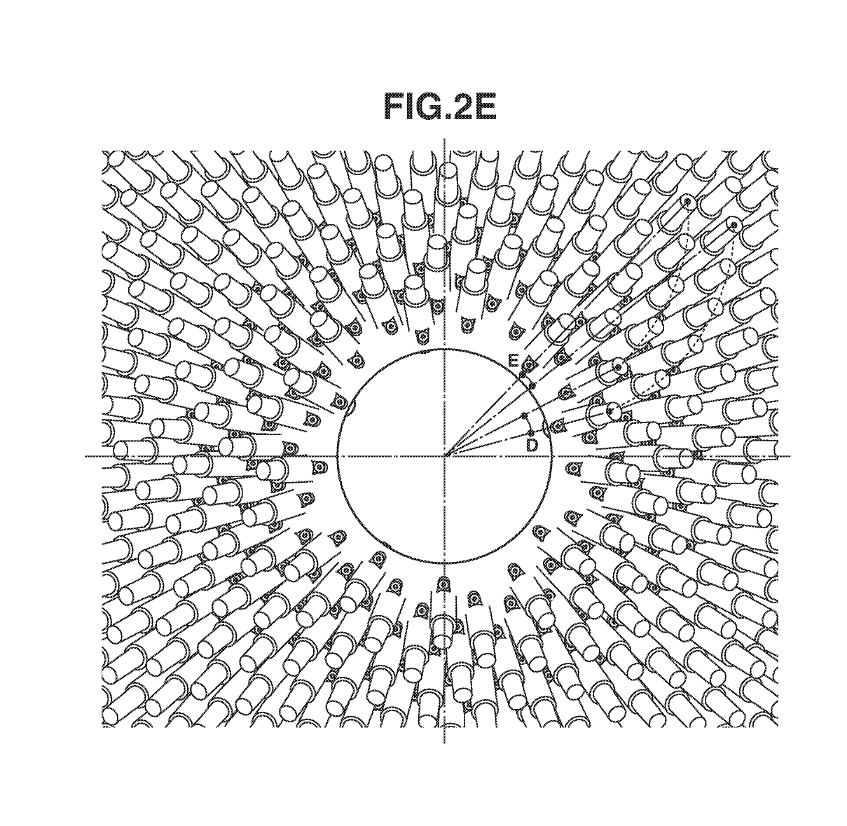

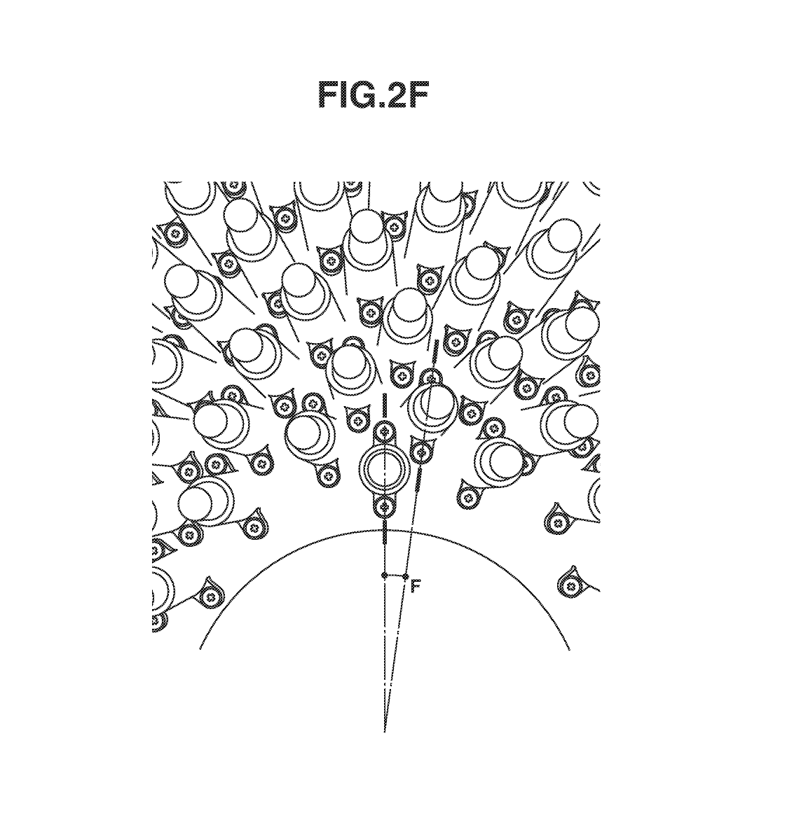

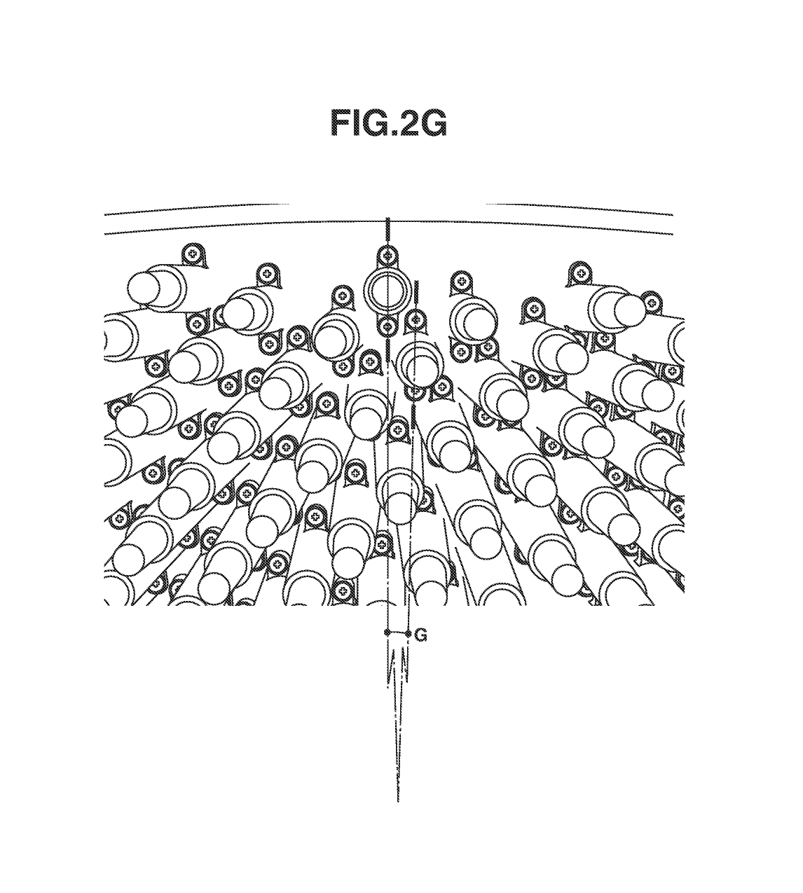

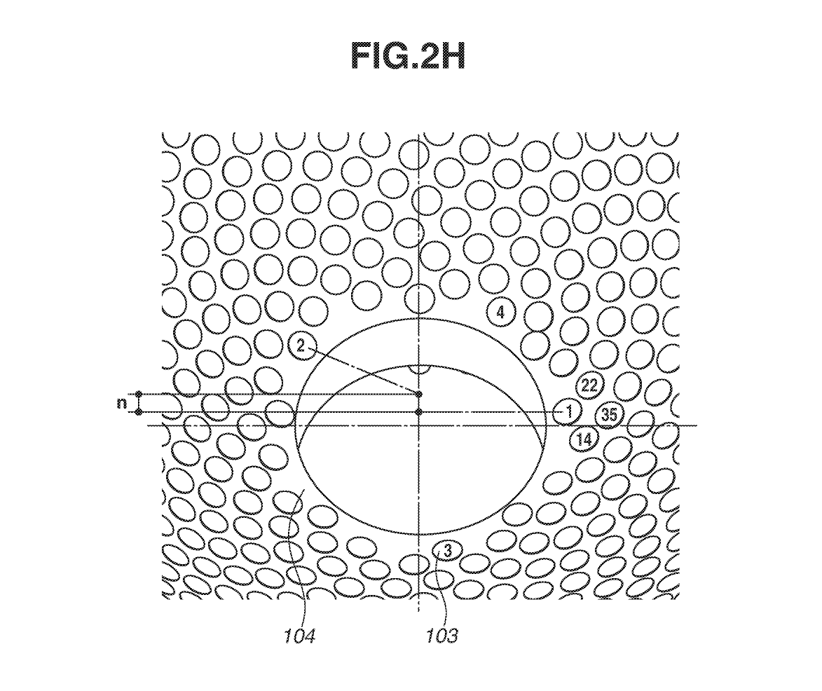

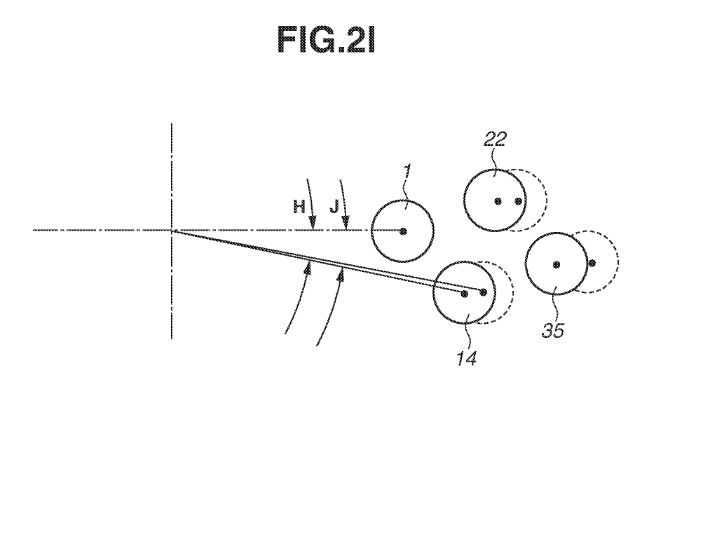

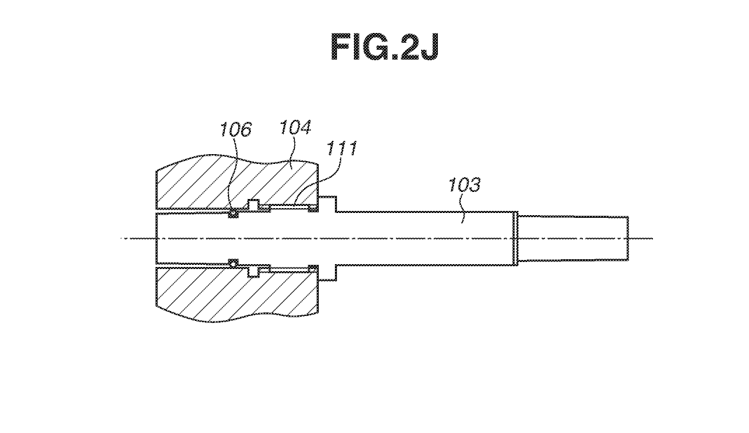

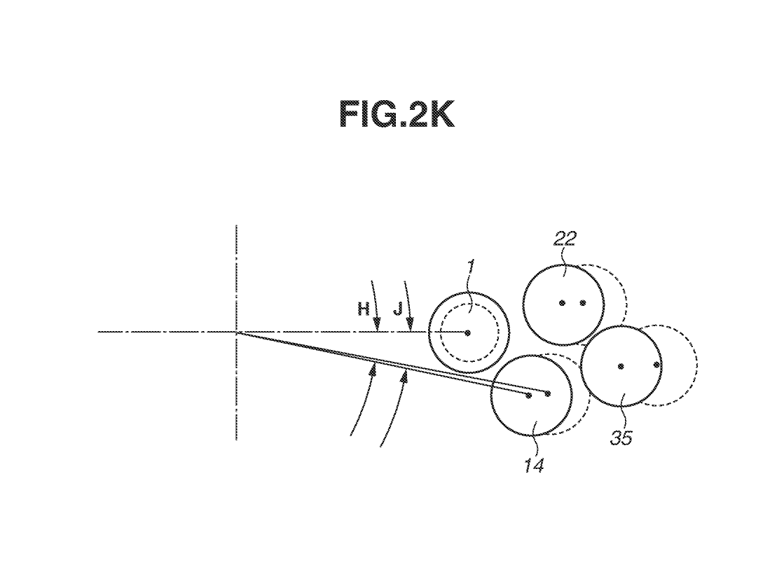

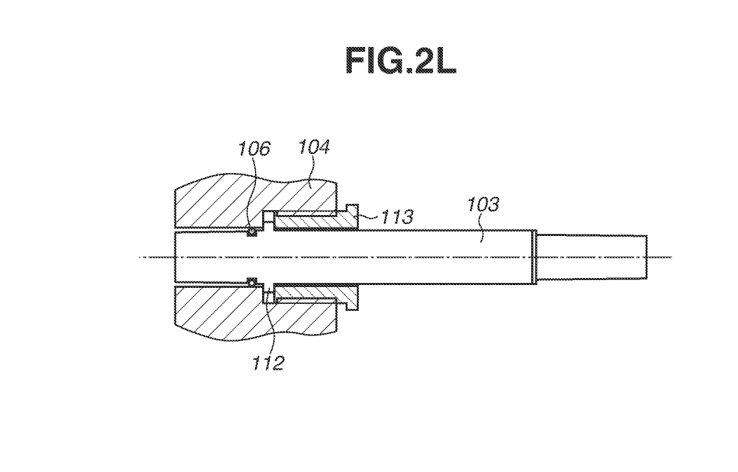

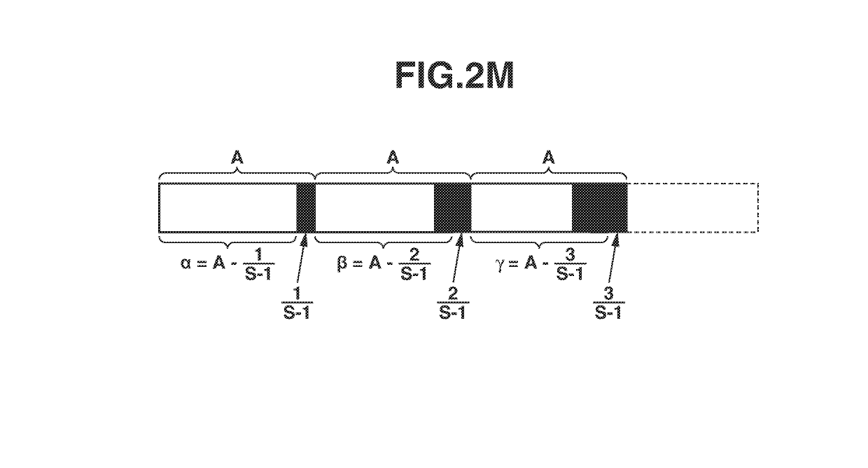

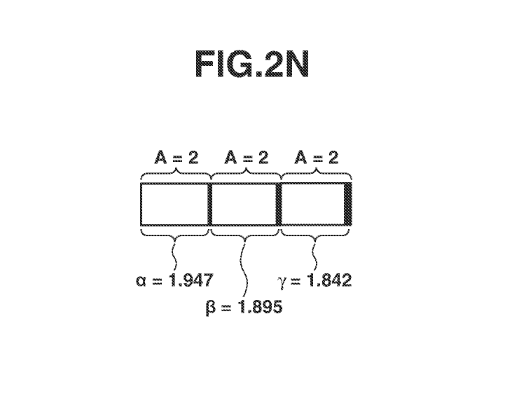

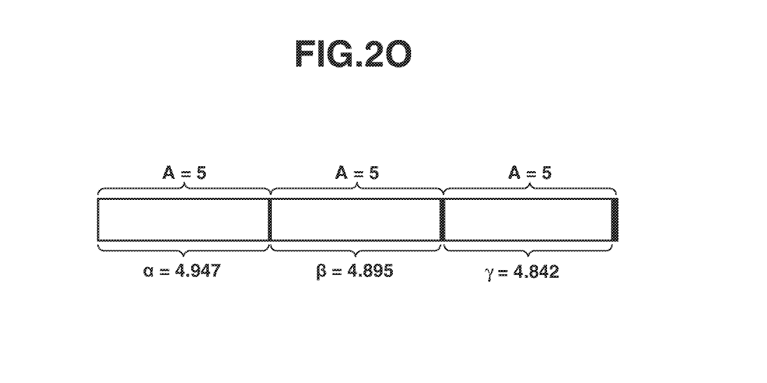

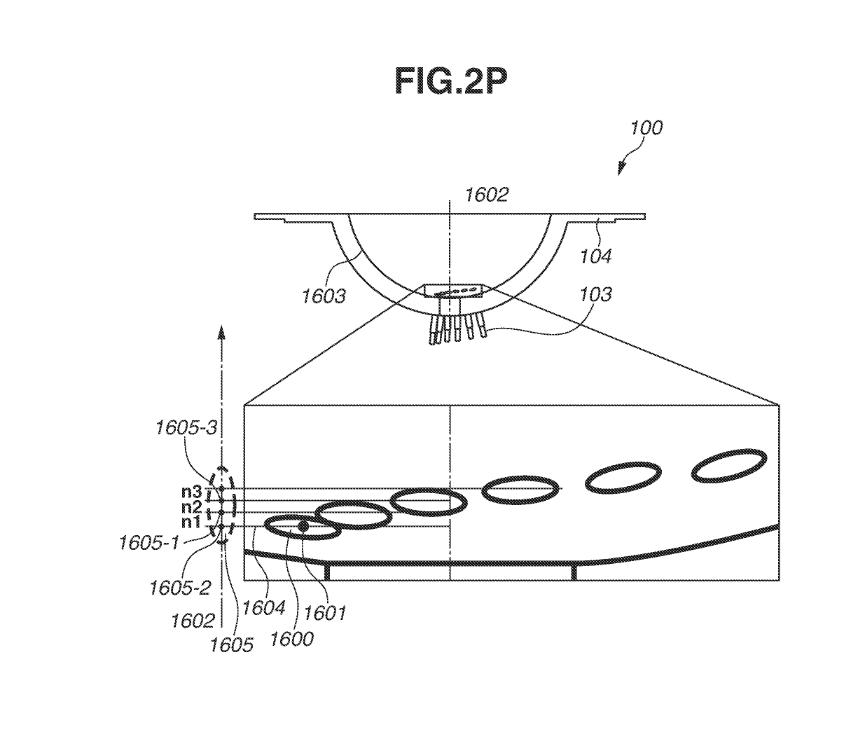

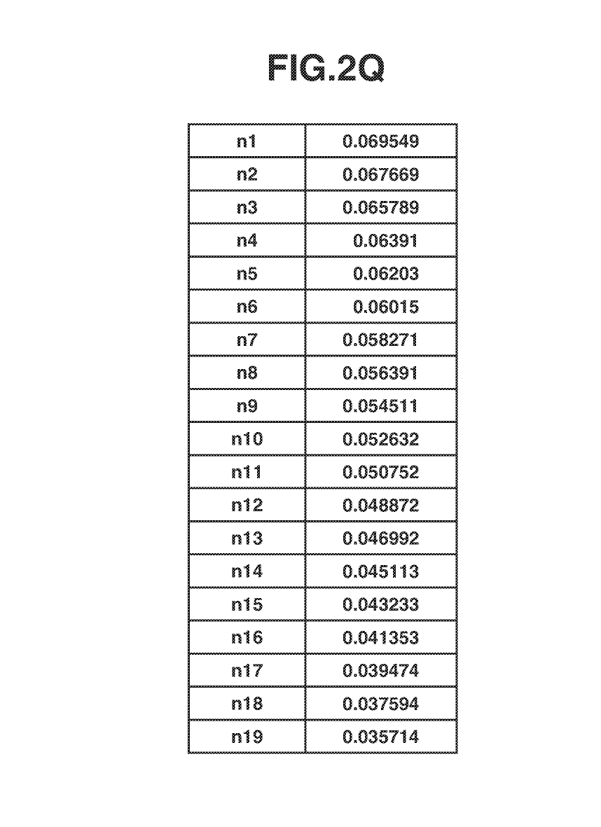

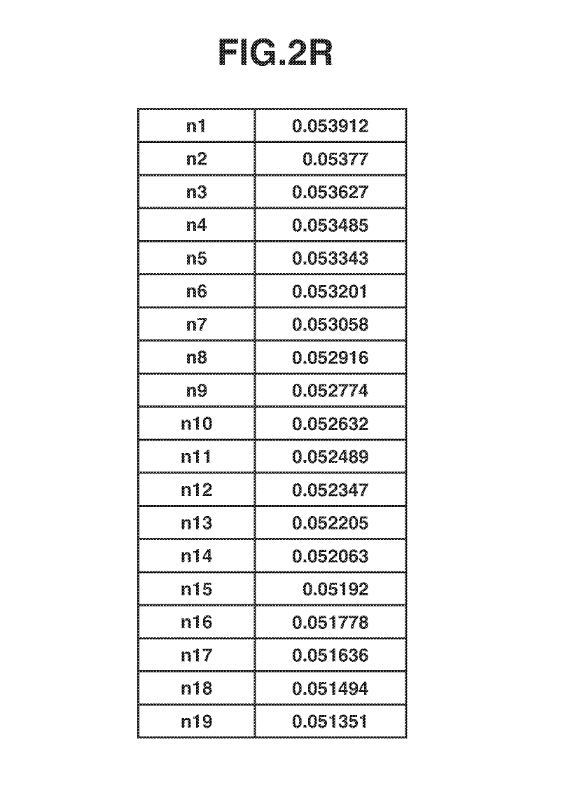

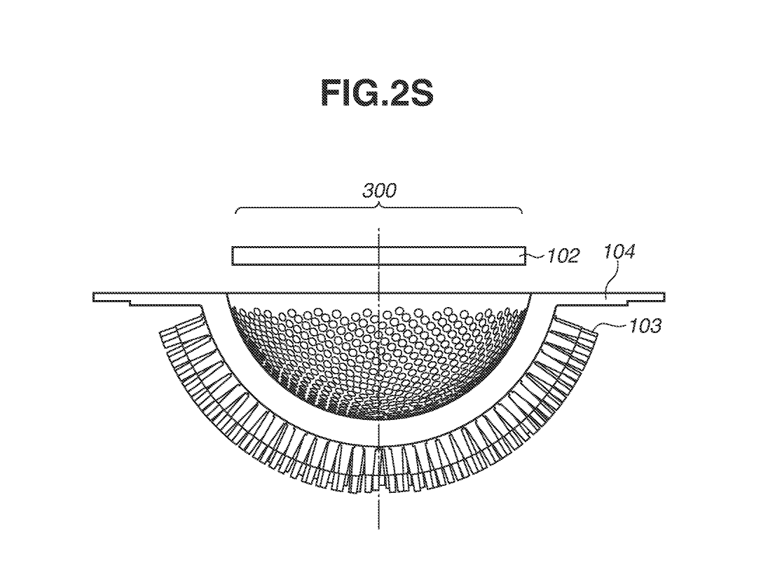

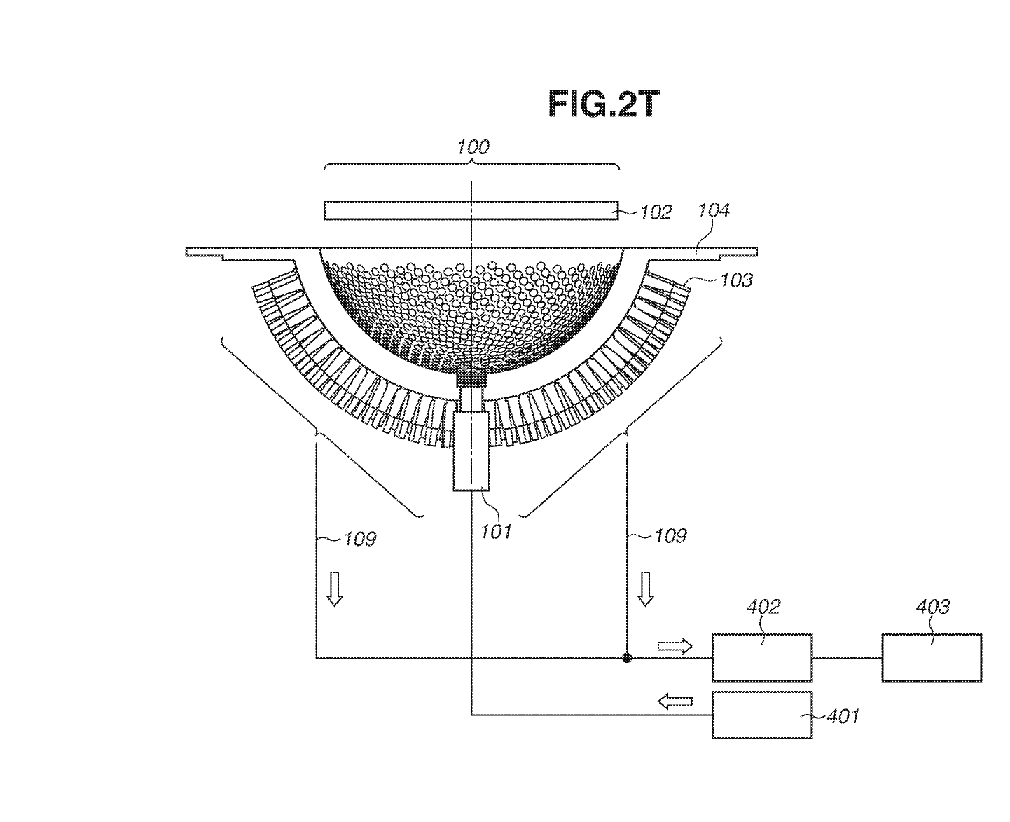

FIG. 2P illustrates a sectional view of a probe array according to the second exemplary embodiment, and an enlarged view thereof.

FIG. 2Q is a chart illustrating specific calculations of differences between the reference points of ith and (i+1)th ultrasonic probes 103 projected upon the axis of rotational symmetry of the housing 104, connecting the bottommost portion of the housing 104 to the subject 102, according to exemplary embodiment 2 of the second exemplary embodiment when the constant A is 2.

FIG. 2R is a chart illustrating specific calculations of differences between the reference points of the ith and (i+1)th ultrasonic probes 103 projected upon the axis of rotational symmetry of the housing 104, connecting the bottommost portion of the housing 104 to the subject 102, according to exemplary embodiment 2 of the second exemplary embodiment when the constant A is 20.

FIG. 2S is a diagram for describing an acoustic wave unit 300 according to exemplary embodiment 3 of the second exemplary embodiment (diagram without the light irradiation unit 101 in FIG. 2A of exemplary embodiment 1 of the second exemplary embodiment).

FIG. 2T is a diagram for describing a subject information acquisition unit according to exemplary embodiment 4 of the second exemplary embodiment.

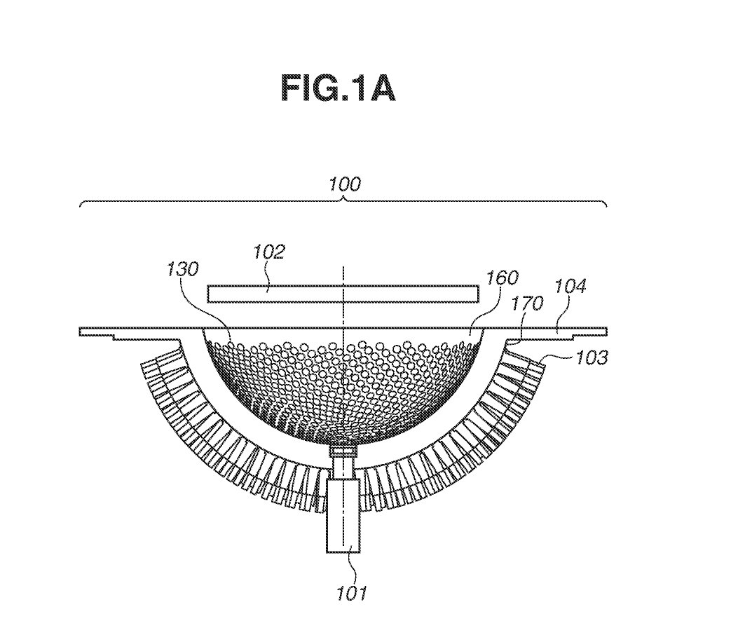

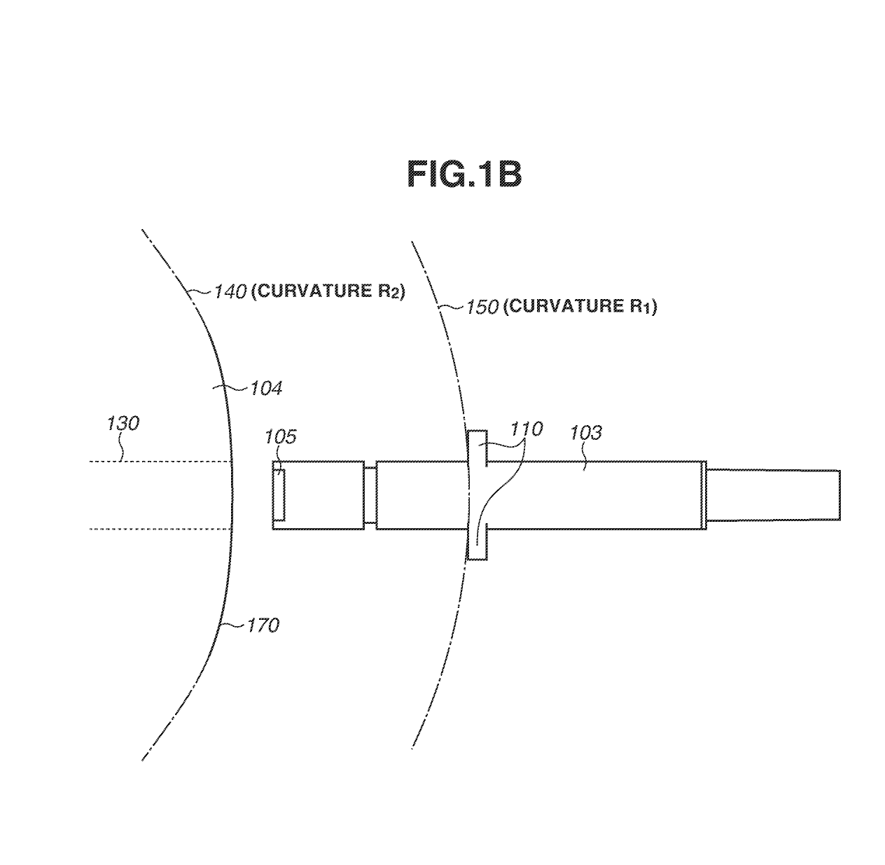

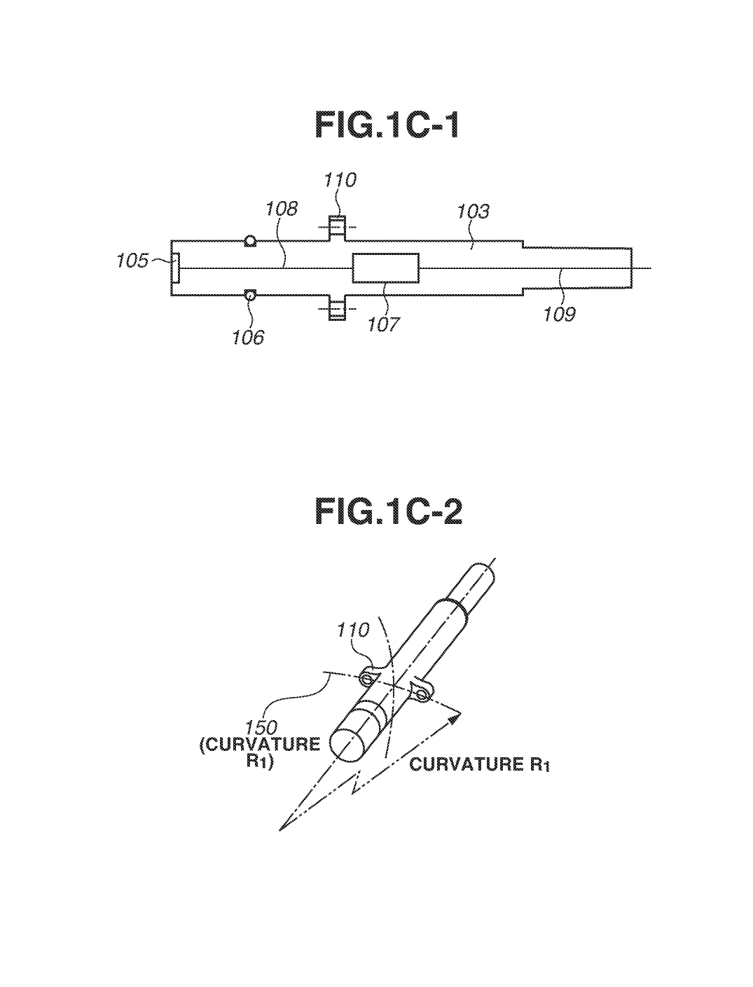

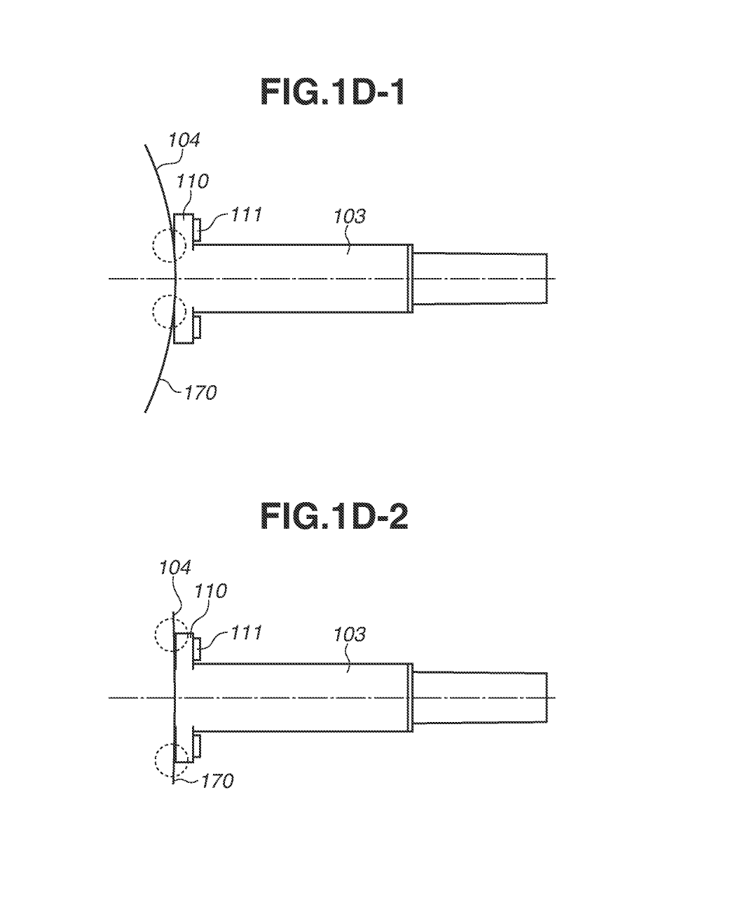

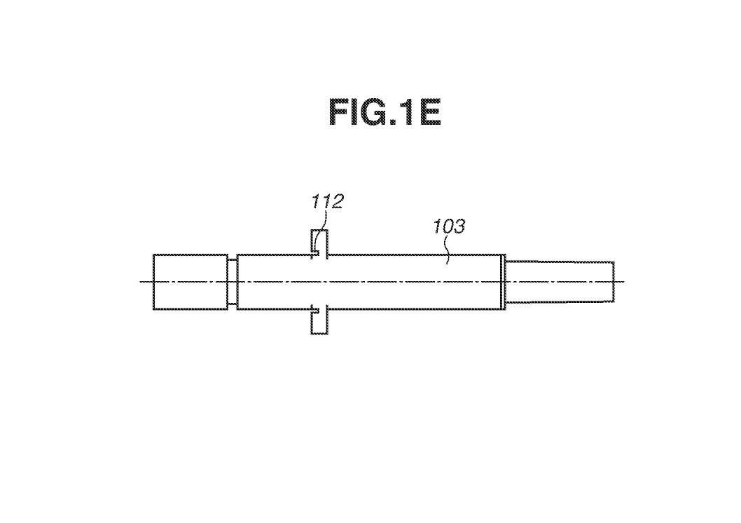

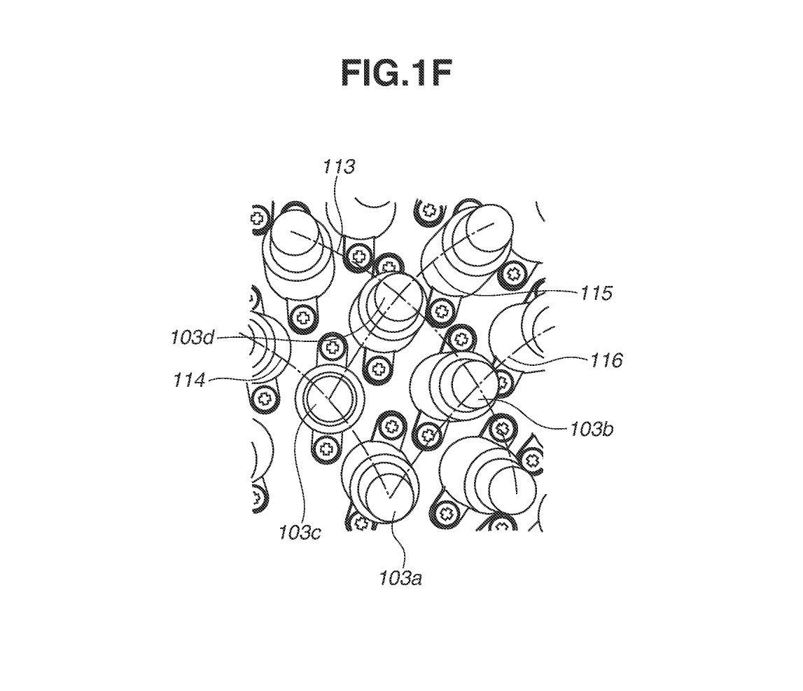

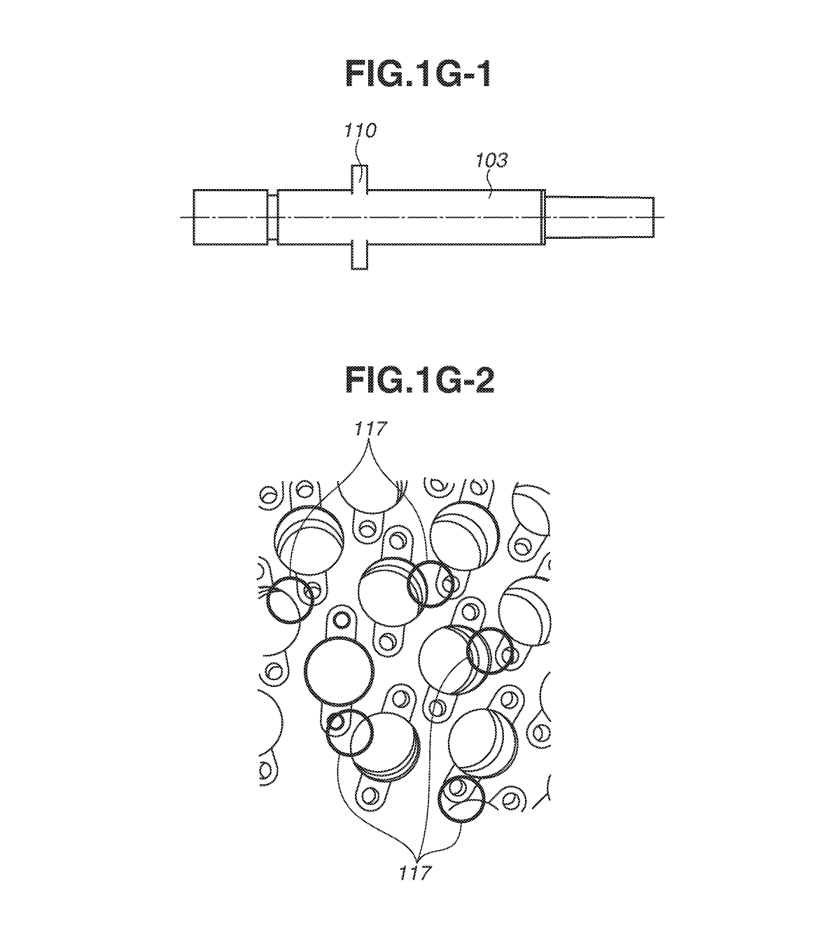

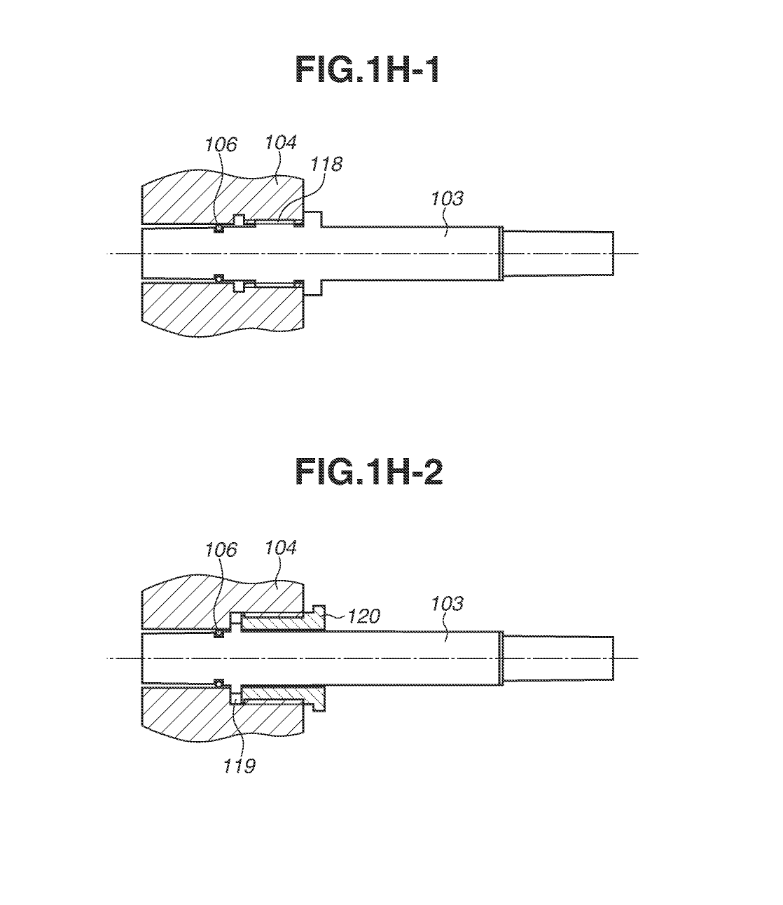

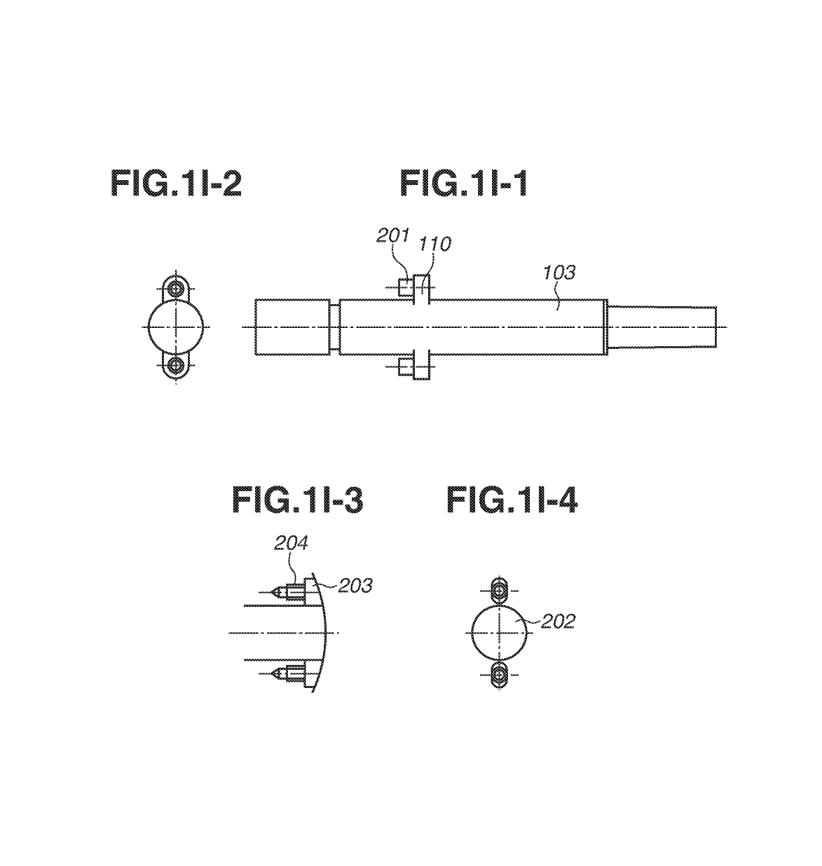

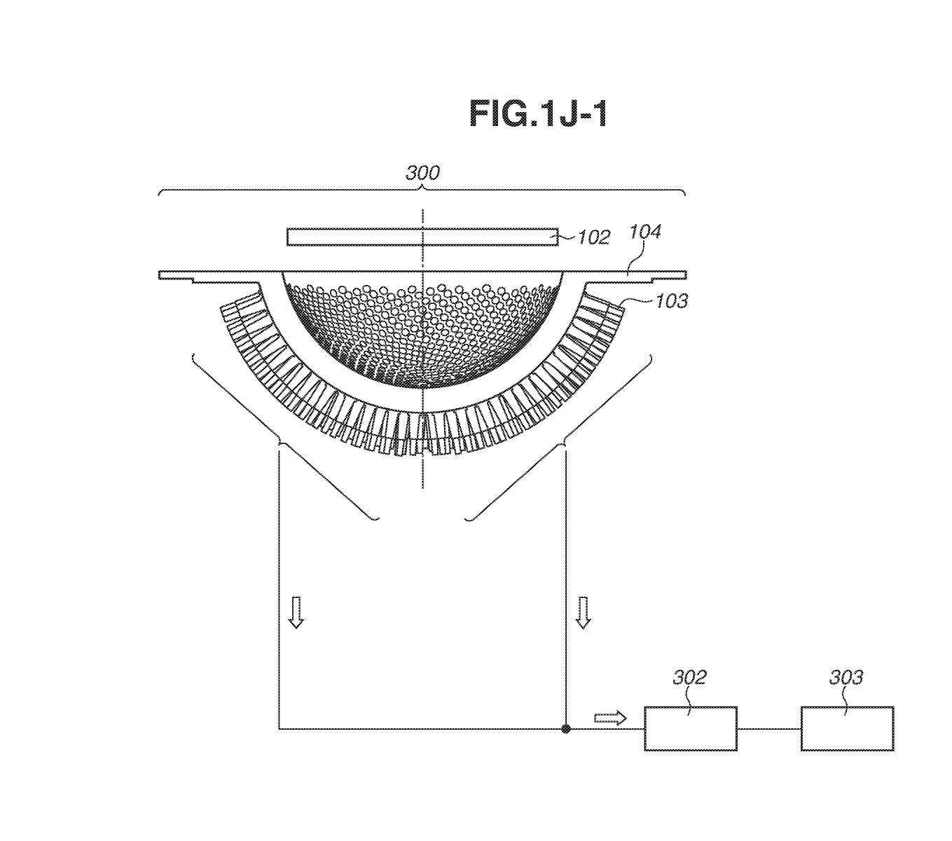

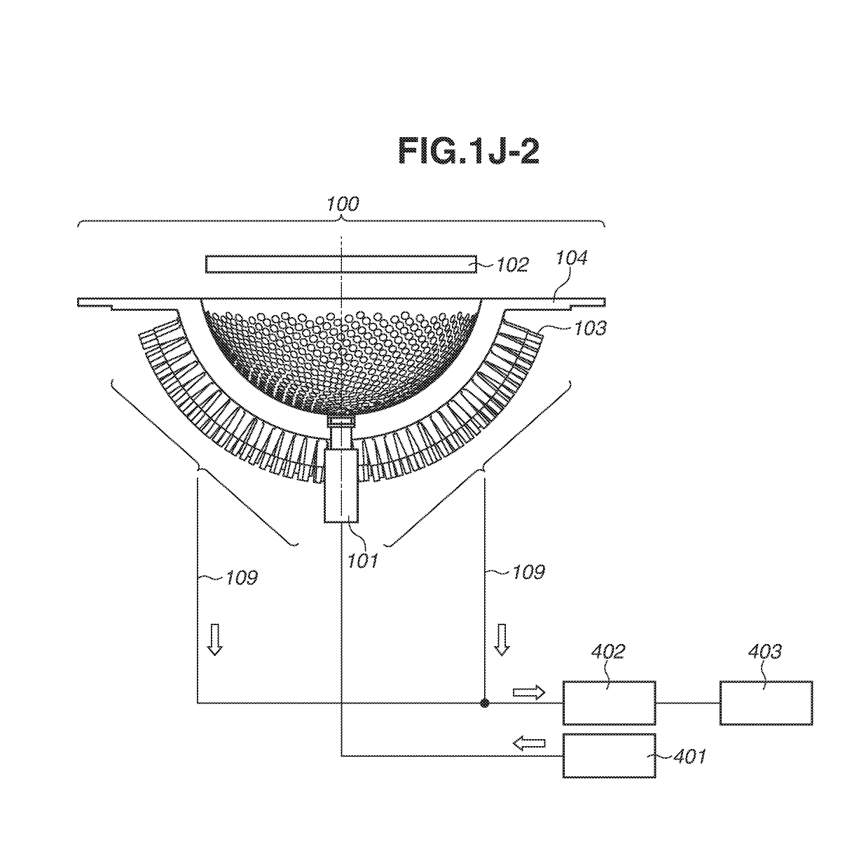

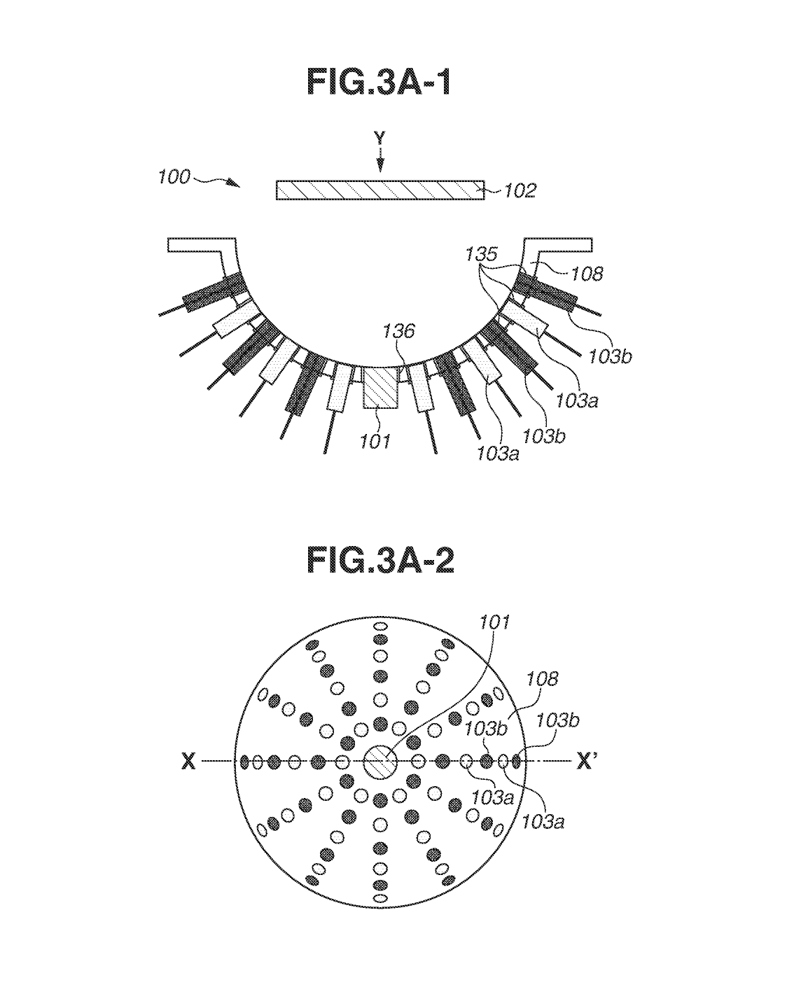

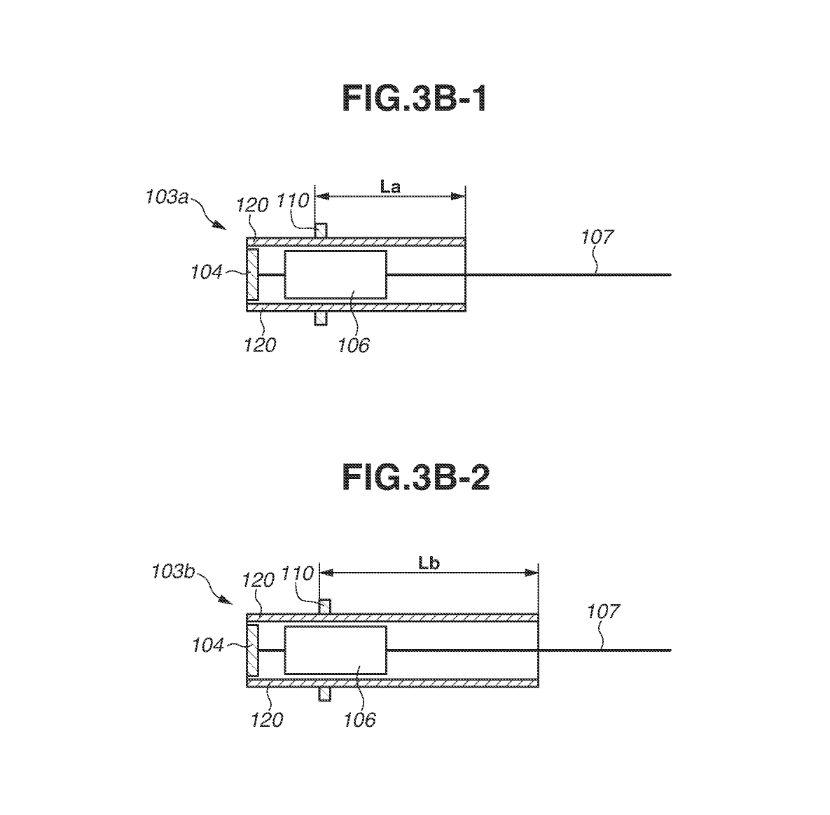

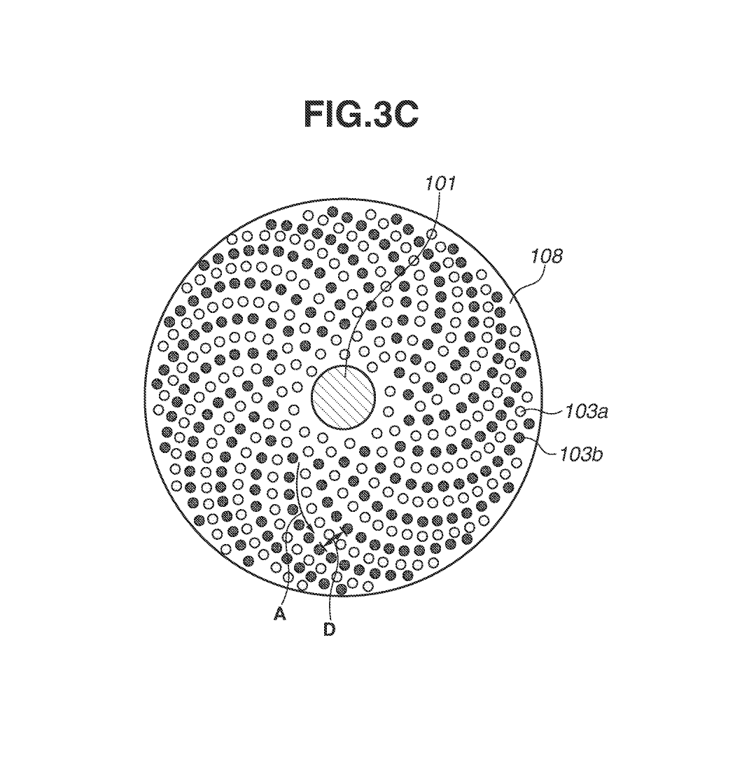

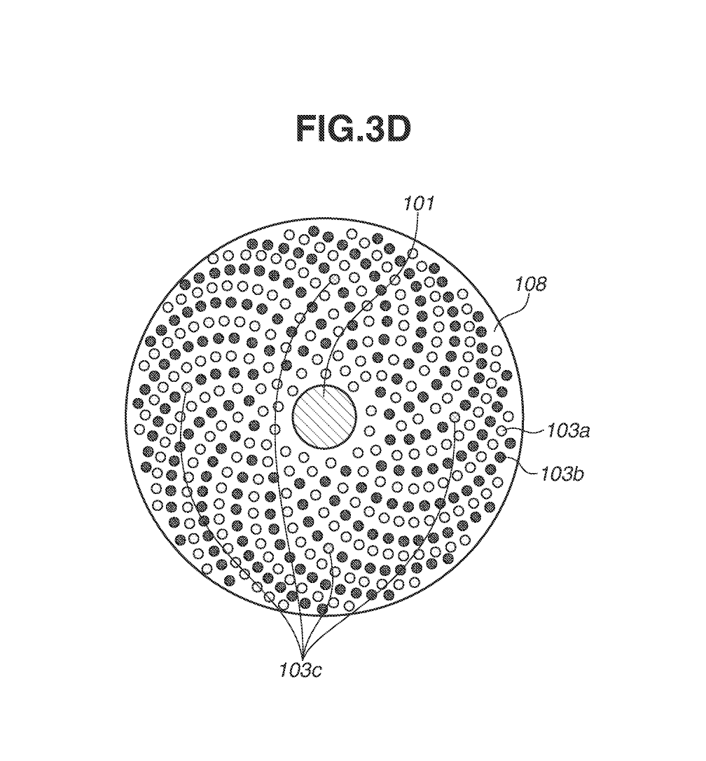

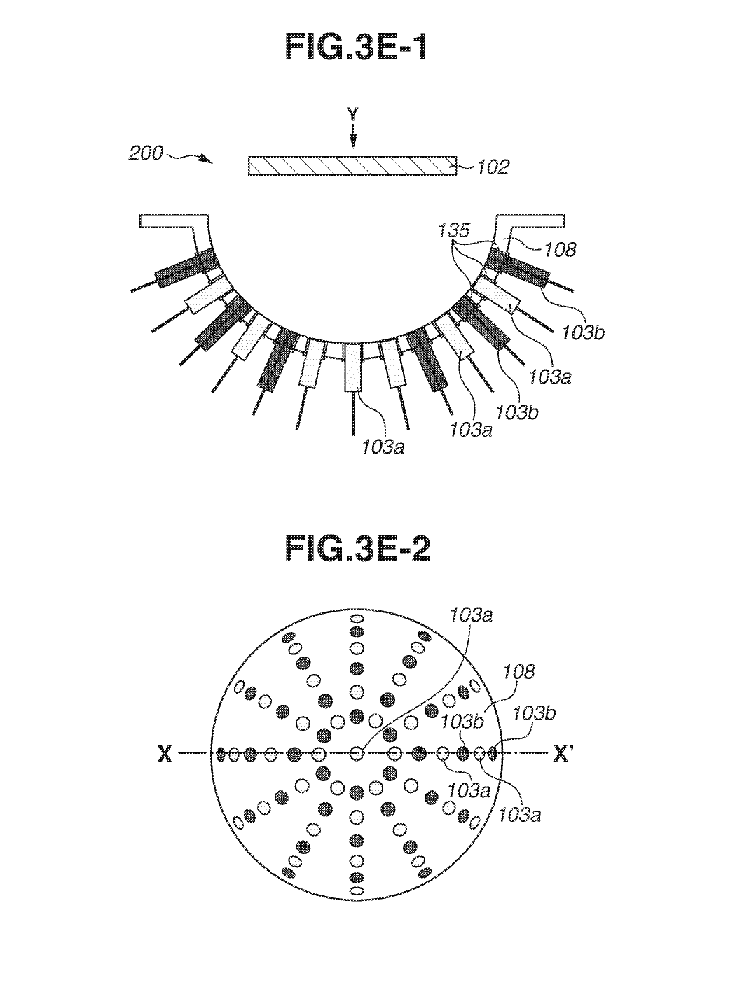

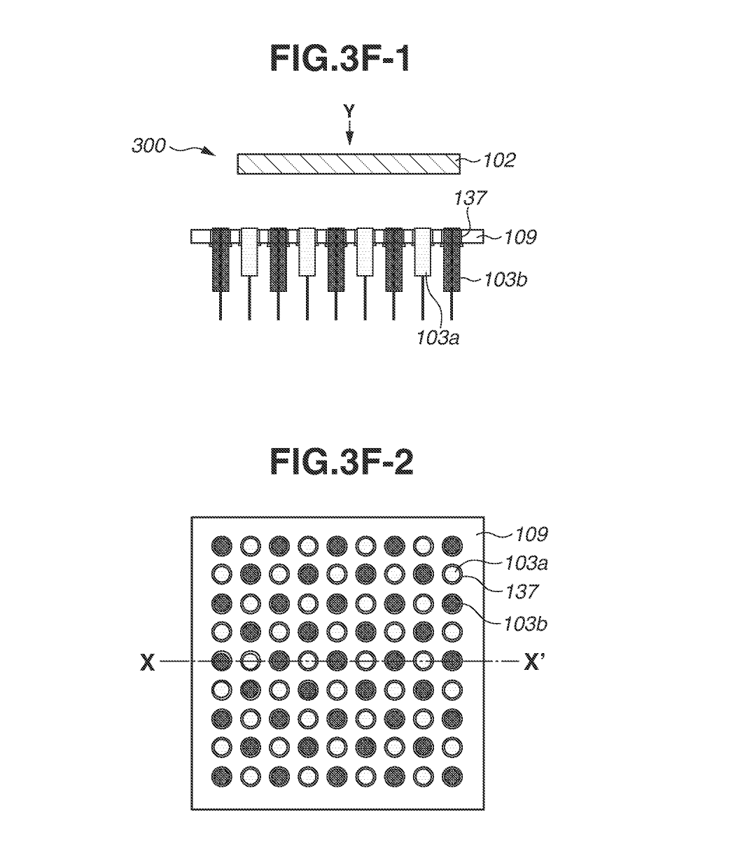

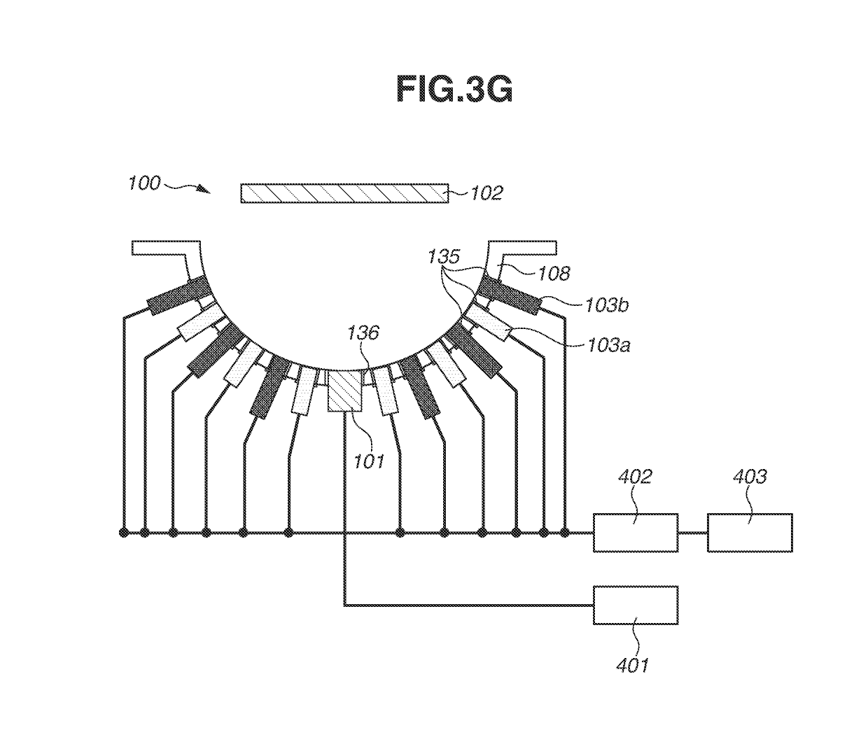

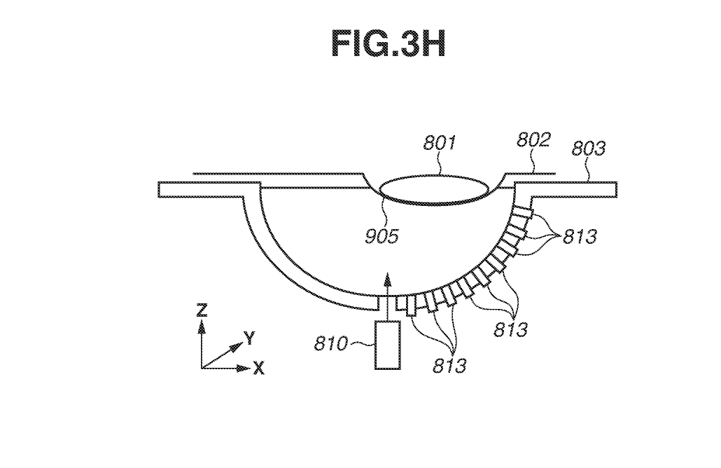

FIG. 3A-1 is a sectional view schematically illustrating an acoustic wave unit according to exemplary embodiment 1 of a third exemplary embodiment.

FIG. 3A-2 is a plan view schematically illustrating the acoustic wave unit according to exemplary embodiment 1 of the third exemplary embodiment.

FIG. 3B-1 is a sectional view schematically illustrating a probe according to exemplary embodiment 1 of the third exemplary embodiment.

FIG. 3B-2 is a sectional view schematically illustrating a probe according to exemplary embodiment 1 of the third exemplary embodiment.

FIG. 3C is a plan view schematically illustrating an acoustic wave unit according to exemplary embodiment 2 of the third exemplary embodiment.

FIG. 3D is a plan view schematically illustrating an acoustic wave unit according to exemplary embodiment 3 of the third exemplary embodiment.

FIG. 3E-1 is a sectional view schematically illustrating an acoustic wave unit according to exemplary embodiment 4 of the third exemplary embodiment.

FIG. 3E-2 is a plan view schematically illustrating the acoustic wave unit according to exemplary embodiment 4 of the third exemplary embodiment.

FIG. 3F-1 is a sectional view schematically illustrating an acoustic wave unit according to exemplary embodiment 5 of the third exemplary embodiment.

FIG. 3F-2 is a plan view schematically illustrating the acoustic wave unit according to exemplary embodiment 5 of the third exemplary embodiment.

FIG. 3G is a schematic diagram illustrating a subject information acquisition unit using the acoustic wave unit according to exemplary embodiment 1 of the third exemplary embodiment.

FIG. 3H is a schematic diagram illustrating an example of a conventional photoacoustic apparatus according to the third exemplary embodiment.

### DESCRIPTION OF THE EMBODIMENTS

First to third exemplary embodiments will be individually described. Configurations including components described in different exemplary embodiments are also intended to be included. Reference numerals and embodiment numbers used in the first to third exemplary embodiments are only effective in the respective exemplary embodiments. For example, reference numeral 100 (probe array) in the first exemplary embodiment refers to ones described in the first exemplary embodiment and ones illustrated in the corresponding drawings (FIGS. 1A to 1J-2), and not the same reference numerals in the second or third exemplary embodiment.

Probe arrays according to the first exemplary embodiment will be described. The invention is not limited thereto.

A probe array according to exemplary embodiment 1 of the first exemplary embodiment will be described with reference to FIGS. 1A and 1B. FIG. 1A is a sectional view for describing a configuration of the probe array according to the present exemplary embodiment. FIG. 1B is an enlarged sectional view of a part of the probe array for describing a curvature (radius of curvature; hereinafter, referred to simply as a curvature) of fixing portions of a probe. For the purpose of description, FIG. 1B illustrates the probe as detached from a support member.

A probe array 100 according to the present exemplary embodiment includes a cup-shaped support member 104 having a plurality of through holes 130, and probes 103 provided in the through holes 130. The probes 103 each include a transducer 105 configured to be capable of conversion between acoustic waves and an electrical signal. The probes 103 each include fixing portions 110 which have a curvature and are intended to fix the probe 103 to an outer surface 170 of the support member 104.

Since the fixing portions 110 of the probes 103 according to the present exemplary embodiment have a curvature, the probes 103 can be fixed along the outer surface 170 of the cup-shaped support member 104. If the probes 103 are arranged on the support member 104 at high density, the outer surface 170 of the support member 104 does not need to be machined into flat shapes according to the fixing portions 110. This can suppress chippings in part of the support member 104 and the occurrence of small ribs and edges due to the machining into flat shapes.

To stably fix the probes 103 to the support member 104 at high density, surfaces 150 of the fixing portions 110 to make contact with the outer surface 170 of the support member 104 desirably have a curvature (R₁) substantially the same as a curvature (R₂) of a surface 140 of the outer surface 170 to make contact with the fixing portions 110. As employed herein, being substantially the same refers to satisfying a relationship of 0.95×R₂≤R₁≤1.05×R₂. The relationship can be desirably 0.99×R₂≤R₁≤1.01×R₂, and more desirably 0.999R₂≤R₁≤1.001×R₂.

Details of the probe array 100 according to the present exemplary embodiment will be described below.

### (Probe Array)

The probe array 100 according to the present exemplary embodiment includes at least the cup-shaped support member 104 having the plurality of through holes 130, and the probes 103 provided in the through holes 130. The probes 103 each include a transducer 105 configured to be capable of conversion between acoustic waves and an electrical signal.

A subject 102 is a part of a human body or animal, such as a hand, a leg, and a breast. The probes (ultrasonic probes) 103 receive acoustic waves occurring from the subject 102. The probes 103 are made of a structural material such as metal and resin. If a plurality of probes 103 is provided on the probe array 100, resin-molded ones are desirably used in consideration of manufacturing cost. The material of the probes 103 may be selected according to that of the support member (housing) 104 to be described below. The plurality of probes 103 is spirally arranged in the respective plurality of through holes 130 of the support member 104. There are various types of spirals, including a logarithmic spiral and the Archimedean spiral. The present exemplary embodiment uses a spiral such that distances between adjoining probes 103 are Fibonacci numbers.

The reception surfaces of the transducers 105 provided in the plurality of probes 103 are directed to inside of an inner surface 160 of the support member 104. The plurality of probes 103 is desirably arranged so that normals to the reception surfaces of the respective transducers 105 intersect at one point. The intersection of the normals is high in sensitivity. If the subject 102 is located at the intersection, highly accurate information about the subject 102 can be obtained.

The probes 103 do not need to be provided for all the through holes 130. Probes 103 as many as according to the subject 102 or information obtained from the subject 102 may be provided.

A light irradiation unit 101 is an optical system for irradiating the subject 102 with pulsed light of the order of nanoseconds. The light irradiation unit 101 is supplied with light from a not-illustrated light source (to be described below).

In the present exemplary embodiment, the support member 104 has a hemispherical shape, and supports the light irradiation unit 101 and the probes 103. Both the inner surface 160 and the outer surface 170 of the support member 104 are hemispherical. The inner and outer surfaces 160 and 170 are desirably shaped, though not limited to, to have the same center of curvature. The curvature of the inner surface 160 and that of the outer surface 170 are desirably substantially the same.

The inner surface 160 does not necessarily need to be hemispherical in shape and may have a parabolic, hyperbolic, elliptic, or polygonal shape, or other shapes as long as the reception of acoustic waves is not interfered. Specifically, the support member 104 may have a substantially hemispherical shape, a truncated conical shape, or a semi-cylindrical shape aside from a hemispherical shape. The substantially hemispherical shape may be such that a line connecting the center of the sphere and the apex of the sphere and a line connecting the center of the sphere and the rim of the sphere form an angle x of smaller than 90° or greater than 90°. If x is 90°, the support member 104 is hemispherical. The center of curvature of the outer surface 170 can be the point at which the foregoing probes 103 are directed and concentrated toward the subject 102. The support member 104 has two types of holes. A first type is a hole for the pulsed light from the light irradiation unit 101 to pass through. Holes of second type are the through holes 130 for the ultrasonic probes 103 to receive acoustic waves, for the probes 103 to be provided in. In the present exemplary embodiment, the center axes of the through holes 130 are directed to the center of curvature of the outer surface 170 of the support member 104. However, such a configuration is not necessarily restrictive.

Next, details of the configuration of a probe 103 will be described with reference to FIGS. 1C-1 and 1C-2. The transducer (acoustic wave conversion element) 105 performs mutual conversion between acoustic waves and an electrical signal. In the present exemplary embodiment, the transducer 105 converts acoustic waves into an electrical signal. A capacitive transducer or a piezoelectric transducer may be used as the transducer 105. A piezoelectric transducer may be made of piezoelectric polymer film material typified by lead zirconate titanate (PZT) or piezoelectric polymer film material typified by polyvinylidene difluoride (PVDF). A capacitive micro-machined ultrasonic transducer (CMUT) may be used as the capacitive transducer.

A sealing member 106 is provide between the support member 104 and the probe 103 so that water and an acoustic matching agent, such as a gel, put in the support member 104 to reduce attenuation of the pulsed light will not leak from the support member 104. In FIG. 1C-1, the sealing member 106 is an O ring. Any member that will not be degraded by water or the matching agent may be used as the sealing member 106. Examples include fluorine-based rubber. Other examples include adhesives. In view of replaceability, an O ring is desirably used as the sealing member 106. In the present exemplary embodiment, one O ring is used, whereas two or more O rings may be provided in consideration of sealability. A circuit 107 processes a signal between the transducer 105 and a not-illustrated system. In the diagram, the circuit 107 is used for signal processing when a signal is received. The circuit 107 may include a circuit for performing signal processing in reversely transmitting a signal. First wiring 108 connects the transducer 105 and the circuit 107. Second wiring 109 connects the signal between the circuit 107 and the not-illustrated system. The fixing portions 110 of the probe 103 are portions to be fixed to the support member 104.

FIG. 1C-2 is a perspective view illustrating the shape of the ultrasonic probe 103 in a perspective manner and illustrating the shape of the fixing portions 110 in detail. In the diagram, the sealing member 106 is not illustrated. The fixing portions 110 have a total of two holes. Not-illustrated fastening members such as screws and bolts are inserted through the holes, whereby the fixing portions 110 are fixed to the support member 104. The two holes are formed in substantially symmetrical positions with the center axis of the cylindrical probe 103 therebetween.

As illustrated in the diagram, the surfaces of the fixing portions 110 to make contact with the support member 104 have the curvature R₁. The curvature R₁ is substantially the same as the curvature R₂ of the outer surface 170 of the support member 104.

The opposite surfaces of the fixing portions 110 are flat surfaces. Normals to the flat surfaces coincide with the center axis of the probe 103. The curvature R₁ is desirably the same as the curvature R₂ of the support member 104, but may be not exactly the same since there usually is manufacturing tolerance. In view of stability of the probe 103, the curvature R₂ of the outer surface 170 of the support member 104 is desirably smaller than the curvature R₁ provided on the fixing portions 110 within the foregoing range of being substantially the same. The reason will be described with reference to FIGS. 1D-1, 1D-2, and 1E.

FIG. 1D-1 is a detailed view when the curvature R₂ of the outer surface 170 of the support member 104 is smaller than the curvature R₁ provided on the fixing portions 110. In FIG. 1D-1, screws or bolts are used as the fastening members 111. As illustrated in FIG. 1D-1, the probe 103 makes contact with the support member 104 at the inner sides of the fixing portions 110. FIG. 1D-2 is a detailed view when the curvature R₂ of the outer surface 170 of the support member 104 is greater than the curvature R₁ provided on the fixing portions 110. As illustrated in FIG. 1D-2, the probe 103 makes contact with the support member 104 at the ends of the fixing portions 110. In the case of FIG. 1D-1, the fixing portions 110 of the probe 103 make contact along the ridge of the outer periphery of the probe 103 (line on which the probe 103 makes contact with the support member 104 in the circled areas of FIG. 1D-1). In contrast, in the case of FIG. 1D-2, the fixing portions 110 make contact at only two points at the ends (corner portions of the probe 103 in the circled areas of FIG. 1D-2). The curvature R₂ of the outer surface 170 of the support member 104 thus is desirably smaller than the curvature R₁ provided on the fixing portions 110. In such a case, the fixing portions 110 makes desirably a moderate deformation. The fixing portions 110 can be brought into closer contact with the support member 104 by determining the thickness of the fixing portions 110 in consideration of the axial force of the fastening members 111 on the fixing portions 110. In this regard, the probe 103 is suitably made of low-strength, easily-deformable resin. For easy deformation, the fixing portions 110 may be notched in part. FIG. 1E is a diagram illustrating the probe 103 of which the fixing portions 110 are notched in part. Notches 112 are made in part of the fixing portions 110. The presence of the notches 112 facilitates the deformation of the probe 103, whereby the closeness of contact of the probe 103 with the support member 104 improves. The thickness of the fixing portions 110 and the size of the notches 112 is desirably determined according to the material of the probe 103 and the shape of the fixing portions 110.

FIG. 1F is an enlarged view seen from the side of the outer surface 170 when the probes 103 are arranged on the support member 104. In the diagram, reference points of adjoining probes 103 are connected by virtual lines 113, 114, 115, and 116. For the purpose of description of the diagram, representative probes 103 will be denoted by 103a, 103b, 103c, and 103d. As illustrated in FIG. 1F, the probes 103 are unevenly arranged. A quadrilateral formed by connecting four reference points of the adjoining probes 103 has a collapsed shape. The distance between the probes 103a and 103b is large, and the distance between the probes 103c and 103d is small.

With such a configuration, for example, the support member 104 can be provided with 600 or more probes 103, and desirably 900 or more probes 103. A probe array 100 including probes 103 arranged at high density can thus be obtained. For example, the probe array 100 according to the present exemplary embodiment can be applied when probes 103 are provide on the hemispherical support member 104 at a density of 2900 probes/m² or more.

### COMPARATIVE EXAMPLE

To describe the reason why the fixing portions 110 are shaped to have a curvature, a comparative example will be described below. FIG. 1G-1 is a diagram for describing a structure when the attaching configuration of the fixing portions 110 to the housing 104 is shaped to be flat without a curvature, as a comparative example of the probe 103. The surfaces of the fixing portions 110 to be attached to the support member 104 are flat surfaces that are parallel with the surfaces to which the not-illustrated fastening members 111 are attached.

FIG. 1G-2 is a diagram illustrating the formation of flat surface portions according to the surfaces of the fixing portions 110 to be attached to the support member 104 so that the probes 103 described in FIG. 1G-1 are arranged on the support member 104. Circles 117 indicate small ribs (hereinafter, referred to as ribs) produced by cutting. As illustrated in FIG. 1G-2, the support member 104 is cut to form flat surfaces according to the shapes of the fixing portions 110. The cutting produces the ribs 117. In the diagram, there are five ribs in mere ten areas or so. As illustrated in FIG. 1G-2, several hundreds of probes 103 are mounted on the support member 104, and there can be a large number of ribs 117.

If the probes 103 are arranged at even higher density, the holes for the probes 103 to be passed through and the flat surface portions formed on the support member 104 can intersect to produce edges. The edges are usually removed not to cut the hands during assembly operations. If more probes 103 are arranged, a large number of ribs make a large number of edges and it takes quite a long time to appropriately dispose of the edges. The fixing portions 110 of the probes 103 can thus be shaped to have a curvature according to that of the support member 104, instead of being flat portions.

### Reference Example 1

FIG. 1H-1 illustrates an alternative structure as an example of the probe 103. Here, the structure of the probe 103 of FIG. 1C-1 will be described to be more suitable than the alternative structure according to the present reference example 1. In FIG. 1H-1, a male screw 118 is formed on the probe 103. The support member 104 is illustrated with hatching. A female screw is similarly formed in the support member 104 to mesh with the male screw 118. As can be seen from FIG. 1H-1, the diameter of the male screw 118 is always greater than that of the sealing member 106. The probe 103 is difficult to be properly positioned since the probe 103 needs to be rotated when mounted on the support member 104.

### Reference Example 2

FIG. 1H-2 illustrates a second alternative structure as an example of the probe 103. In this example, the structure of probe 103 of FIG. 1C-2 will be described to be more suitable than the second alternative structure. In FIG. 1H-2, an abutting portion 119 is provided on the outer periphery of the probe 103. The abutting portion 119 is abutted against an abutting surface provided inside the support member 104. A press ring 120 is intended to press the abutting portion 119 of the probe 103. A male screw is formed on the outer periphery of the press ring 120. A female screw is formed in the support member 104 to mesh with the male screw. In the alternative structure described in FIG. 1H-1, the probe 103 is rotated when mounted on the support member 104. In the second alternative structure of FIG. 1H-2, the probe 103 is inserted until the abutting portion 119 is abutted against the support member 104, and the press ring 120 is screwed in for fixing. Although not illustrated in FIG. 1H-2, if the abutting portion 119 includes an orientation-determining mechanism, such as a D-cut in part of the probe 103, and the support member 104 is machined accordingly, the orientation of the probe 103 can also be determined. However, since the second alternative structure is configured so that the probe 103 is pressed and fixed by the press ring 120, the diameter of the press ring 120 is always greater than that of the sealing member 106.

In both the alternative structures of the probe 103 illustrated in FIGS. 1H-1 and 1H-2, the screw portion or the press ring 120 has a diameter greater than that of the sealing member 106. For arrangement at even higher density, the structure of the probe 103 illustrated in FIG. 1C-2 is therefore suitable. As described above, the probe 103 can also be fixed by using an adhesive, whereas adhesives are better not used in view of replaceability.

The occupation area of the probes 103 can be further increased by using a single fastening member 111 to fasten a fixing portion 110 of each probe 103. However, since the fastening member 111 for pressing the fixing portion 110 of the probe 103 is positioned off the center axis of the ultrasonic probe 103, the probe 103 makes an uneven contact and can be fixed obliquely to the hole of the support member 104. The two-point fixing as in the present structure is therefore more desirable.

The holes for fixing the fixing portions 110 of the probe 103 are formed in symmetrical positions (opposite positions) with the center axis of the probe 103 therebetween. Either one of the fixing portions 110 may be provided in a somewhat rotated position to effectively accommodate an irregular arrangement. However, since the opposition of the two fastening members 111 with the center axis therebetween enables axial alignment, the fixing portions 110 are desirably opposed as long as possible. If a sufficient area is available, the probe 103 is desirably supported at three points, which may be contradictory to high density mounting.

In FIG. 1F, the probes 103 are directed with their fixing portions 110 vertical on the diagram. However, the probes 103 may be rotated in any angle to avoid interference. For example, in the diagram, the probes 103 may be rotated by 90° about their rotation axes. Every other probe 103 may be changed in angle. The probes 103 can be arranged at high density by directing the probes 103 in optimum angles.

As described above, the fixing portions 110 of the probes 103 are shaped to have a curvature according to the support member 104, and configured to be fixed by two fastening members 111. The probes 103 are thereby arranged on the support member 104 at high density. This enables image acquisition with high accuracy. The support member 104 does not produce small ribs or edges, and the probes 103 can be safely mounted on the support member 104 during assembly operations. Since the support member 104 does not produce small ribs or edges, machining for removing the ribs or edges is not needed. This can reduce the manufacturing cost of the support member 104.

A configuration for obtaining an image with high accuracy by using a probe array according to exemplary embodiment 2 will be described. A description of similarities to exemplary embodiment 1 will be omitted. Differences will be described below.

The fixing portions 110 of the probe 103 described in exemplary embodiment 1 have a total of two holes, and the fastening members 111 are inserted into the holes for fixing. The two holes have some clearance for inserting the fastening members 111. The probe 103 can thus rotate axially as much as the clearance. To obtain an image with higher accuracy, high precision positioning is needed.

FIG. 1I-1 is a diagram in which positioning mechanisms are provided on the fixing portions 110 for fixing the probe 103 to a predetermined position of the support member 104. FIG. 1I-2 is a side view of FIG. 1I-1. In FIG. 1I-1, positioning mechanisms 201 are two doughnut-like cylinders provided on the mounting surfaces of the fixing portions 110 on the support member 104 side. FIG. 1I-3 is a diagram for describing a hole structure on the support member 104 side. FIG. 1I-4 is a side view of FIG. 1I-3.

In FIG. 1I-3, a hole 202 is a hole for the probe 103 to be passed through. Long holes 203 are formed according to the positioning mechanisms 201 of the fixing portions 110. The long holes 203 and the positioning mechanisms 201 are configured to be fittable to each other, and the rotation of the probe 103 is suppressed by such fitting portions. Tapped holes 204 are intended to fix the not-illustrated fastening members 111. The positioning mechanisms 201 are inserted into the long holes 203 and fixed by the fastening members 111, whereby the axial rotation of the probe 103 can be suppressed to obtain an image with higher reliability. Since the machining on the support member 104 side is relatively simple, the probe array 100 can be manufactured at low cost. While FIG. 1I-1 illustrates the configuration in which the positioning mechanisms 201 are provided on the fixing portions 110, a similar effect can be obtained by forming the holes in the fixing portions 110 with high precision and providing the fastening members 111 with a mechanism for fitting to the holes.

As described above, the provision of the positioning mechanisms 201 on the fixing portions 110 of the probe 103 enables positioning of the probe 103. The probes 103 can thus be mounted at high density, and an image can be obtained with higher accuracy.

### (Information Acquisition Apparatus)

Exemplary embodiment 3 will be described. FIG. 1J-1 is a diagram illustrating an information acquisition apparatus (subject information acquisition apparatus) which includes a probe array 300 without the light irradiation unit 101 of FIG. 1A according to the foregoing exemplary embodiment 1. Similar components to those of exemplary embodiment 1 are designated by the same reference numerals. A description thereof will be omitted. The arrows in FIG. 1J-1 represent a flow of pulsed light, acoustic waves, or signals. The information acquisition apparatus according to the present exemplary embodiment may use the probe array 100 according to exemplary embodiment 2.

In the present exemplary embodiment, the probe array 300 is configured to be capable of transmitting ultrasonic waves. In FIG. 1J-1, the probes 103 include the transducers 105 as described in exemplary embodiment 1. By using the transducers 105, the probes 103 not only receive but also transmit acoustic waves (ultrasonic waves). Specifically, electrical signals for transmission are transmitted from a not-illustrated system to the transducers 105 and converted into acoustic waves. The converted acoustic waves are emitted to the subject 102 and reach the subject 102. The reached acoustic waves are reflected by the subject 102, and converted into electrical signals again by the transducers 105 in the probes 103. The electrical signals are transmitted to an information acquisition unit 302. The information acquisition unit 302 processes the output electrical signals to obtain information about the subject 102. In that sense, the information acquisition unit 302 may be referred to as a signal processing unit. A display unit 303 displays the information about the subject 102, obtained by the information acquisition unit 302. The use of the probe array 300 according to the present exemplary embodiment allows the display unit 303 to display highly accurate information about the subject 102.

The information acquisition unit 302 according to the present exemplary embodiment generates data related to optical characteristic value distribution information such as an absorption coefficient distribution in the subject 102 by using the received electrical signals. When calculating the absorption coefficient distribution in the subject 102, the information acquisition unit 302 typically calculates an initial sound pressure distribution in the subject 102 based on the received electrical signals. The information acquisition unit 302 then calculates the absorption coefficient distribution in consideration of light fluence in the subject 102. The initial sound pressure distribution can be formed, for example, by using back projection in a time domain.

The relationship between the probes 103 and the support member 104 is the same as in FIG. LA. As described above, exemplary embodiment 3 can provide similar effects to those described in exemplary embodiment 1.

### (Information Acquisition Apparatus)

Exemplary embodiment 4 will be described. Here, another example of the information acquisition apparatus (subject information acquisition apparatus) according to the first exemplary embodiment will be described. A description of similarities to exemplary embodiment 3 will be omitted. Differences will be described below.

FIG. 1J-2 is a diagram illustrating that the probe array 100 described in exemplary embodiment 1 is used and applied to an information acquisition apparatus. In FIG. 1J-2, part of exemplary embodiment 1 is applied. Exemplary embodiment 2 is also applicable. In FIG. 1J-2, components similar to those of exemplary embodiments 1 and 2 are designated by the same reference numerals. A description thereof will be omitted. The arrows in the diagram represent a flow of pulsed light, acoustic waves, or signals.

A light source 401 emits pulsed light of the order of nanoseconds. The light source 401 can be selected according to the subject 102. For example, if the subject 102 is a living body, a light source 401 having a wavelength of approximately 600 nm to 1500 nm may be selected. The pulsed light emitted from the light source 401 is passed through the light irradiation unit 101 to irradiate the subject 102. The subject 102 absorbs light energy from the irradiated pulsed light and expands instantaneously to generate acoustic waves. The generated acoustic waves are captured by the plurality of probes 103, and signal intensities and phase information are transmitted to an information acquisition unit 402. The information acquisition unit 402 reconstructs an image based on position information about the ultrasonic probes 103 and the obtained signals. A display unit 403 displays the image.

As described above, the subject 102 is irradiated with the pulsed light generated from the light source 401. Elastic waves occurring from the subject 102 are captured by the probes 103, and image reconstruction is performed based on the signals obtained from the probes 103 and the position information about the probes 103. By the image reconstruction, the information about the subject 102 can be reconstructed as an image.

Exemplary embodiments 3 and 4 differ in the presence or absence of the light irradiation unit 101. Exemplary embodiment 4 is the same as exemplary embodiment 3 in that the received signals are transmitted to the information acquisition unit 402 and then displayed on the display unit 403.

### (Light Source)

The light source 401 according to the present exemplary embodiment will be described in detail. The light source 401 according to the present exemplary embodiment emits pulsed light having a wavelength to be absorbed by a specific component among components constituting a living body. The wavelength used in the present exemplary embodiment is desirably such that the light propagates into the subject 102. Specifically, if the subject 102 is a living body, the wavelength can be longer than or equal to 600 nm and shorter than or equal to 1500 nm. For effective generation of photoacoustic waves, a pulse width of approximately 10 to 100 nanoseconds is suitable. A high-output laser is desirably used as the light source 401. A light-emitting diode (LED) or a flash lamp may be used instead of the laser. Various lasers can be used, including a solid laser, a gas laser, a dye laser, and a semiconductor laser. Irradiation timing, a waveform, and intensity are controlled by a light source control unit. The light source control unit may be integrated with the light source 401. The light source 401 may be provided as a member separate from the information acquisition apparatus according to the present exemplary embodiment. The light source 401 according to the present exemplary embodiment may be one that can emit a plurality of wavelengths of light.

### Background and Problem of Second Exemplary Embodiment

A second exemplary embodiment will be described. WO 2010/030817 discusses a photoacoustic apparatus including a hemispherical acoustic wave detector on which a plurality of acoustic elements is spirally arranged, and a hemispherical container which accommodates a region to be inspected of a subject. In the photoacoustic apparatus, the hemispherical acoustic wave detector is arranged under the container. A light irradiation unit for irradiating the region to be inspected with light is provided in a lower part of the hemispherical acoustic wave detector. With such an apparatus configuration, acoustic waves from the region to be inspected are received in multiple directions, whereby the resolution of the obtained information image is improved.

U.S. Pat. No. 5,713,356 discusses an example of a photoacoustic apparatus similar to that of WO 2010/030817. The photoacoustic apparatus includes a plurality of acoustic elements spirally arranged on a hemispherical acoustic wave detector.

To obtain highly reliable image quality, the probes including the transducers need to be arranged adjacent to each other and mounted at high density. However, neither of U.S. Pat. No. 5,713,356 and WO 2010/030817 discusses a method for fixing the probes including the transducers to the cup-shaped (hemispherical) container at high density.

The second exemplary embodiment is directed to the provision of a probe array in which probes including transducers for transmitting and receiving ultrasonic waves can be arranged on a cup-shaped housing at high density. According to the probe array according to the second exemplary embodiment, the probes including the transducers can be arranged on a cup-shaped support member at high density.

### Detailed Description of Second Exemplary Embodiment

The probe array according to the second exemplary embodiment will be described with reference to FIG. 2P. An upper part of FIG. 2P is a sectional view of the probe array, below which a partial enlarged view of the sectional view is illustrated.

A probe array 100 according to the present exemplary embodiment includes a cup-shaped support member 104 having a plurality of holes 1600, and cylindrical probes 103 provided in the holes 1600. The probes 103 each include a transducer (not illustrated) configured to be capable of conversion between acoustic waves and an electrical signal. A curved surface 1603 including an inside surface of the cup-shaped support member 104 intersects with center axes (center axes of rotation (FIG. 2C) to be described below) of the cylindrical probes 103 at points 1601. An axis of rotational symmetry 1602 of the cup-shaped support member 104 and normals 1604 from the respective points 1601 to the axis of rotational symmetry 1602 have a plurality of intersections 1605. The probe array 100 according to the present exemplary embodiment is characterized by the positional relationship between the plurality of intersections 1605. More specifically, a distance between a first intersection among the plurality of intersections 1605 and a second intersection adjoining the first intersection on an apex side of the cup-shaped support member 104 is greater than a distance between the first intersection and a third intersection adjoining the first intersection on a side opposite from the apex side. In FIG. 2P, suppose that a distance between a first intersection 1605-1 and a second intersection 1605-2 is n1, and a distance between the first intersection 1605-1 and a third intersection 1605-3 is n2. Here, n1 is greater than n2. With such a configuration, the plurality of probes 103 can be arranged on the support member 104 at high density.

A distance between adjoining intersections among the plurality of intersections 1605 may decrease in a direction away from the apex on the axis of rotational symmetry 1602. Some of the distances between adjoining intersections among the plurality of intersections 1605 may be the same. As employed herein, the apex side refers to the bottom side of the support member 104. The direction away from the apex on the axis of rotational symmetry 1602 refers to the direction of the arrow 1602 in FIG. 2P. In the present exemplary embodiment, the distances between adjoining intersections among the plurality of intersections 1605 have a relationship of n1>n2>n3. Some of the distances may be the same.

Suppose that the plurality of intersections 1605 is numbered from 1 to s (s is the total number of probes 103) in order in the direction away from the apex on the axis of rotational symmetry 1602. The plurality of probes 103 may be provided so that a distance ni between an ith intersection (1<i<s; i is a positive integer) and an (i+1)th intersection satisfies the following expression:

$$ni = \frac{( A - ( \frac{i}{s - 1} ) )}{{\sum }_{i = 1}^{s - 1} ( A - ( \frac{i}{s - 1} ) )} \times ( Z max - Z min ) \qquad ( 3 )$$

In the foregoing expression (3), A is a constant greater than or equal to 1, Zmin is a distance from the apex to an intersection located closest to the apex among the plurality of intersections 1605, and Zmax is a distance from the apex to an intersection located farthest from the apex among the plurality of intersections 1605.

The probes 103 may be arranged to protrude from an outside surface of the cup-shaped support member 104. Protruded structures can be used as fixing portions to the support member 104.

Sealing members are provided between the probes 103 and the support member 104, whereby intrusion of a liquid can be suppressed.

The probes 103 are not limited in particular. Capacitive transducers or piezoelectric transducers may be used. Capacitive transducers are called CMUTs, and can transmit and receive up to high-bandwidth ultrasonic waves (acoustic waves).

An information acquisition apparatus can be configured to include the probe array 100 according to the present exemplary embodiment and an information acquisition unit that obtains information about a subject at least based on electrical signals obtained by the probes 103.

Exemplary embodiment 1 will be described. Spiral arrangements, including one using the Fibonacci sequence, are methods by which probes including transducers can be arranged on a cup-shaped support member at high density. Since the probes are unevenly arranged, the probes need to be arranged so that mechanical portions do not interfere during fixing.

If probes including acoustic elements are arranged on a hemispherical container, the bottom side of the hemispherical container has a small mounting area since the distance from the axis of symmetry passing through the center of gravity of the hemispherical container is small. There has thus been a problem of interference between the probes. It has therefore been difficult to mount the probes at high density.

A probe array according to the present exemplary embodiment solves such a problem. Details will be described below.

FIG. 2A is a sectional view illustrating a configuration of an acoustic wave unit (probe array) 100 according to exemplary embodiment 1.

A light irradiation unit 101 is an optical system for irradiating a subject 102 to be described below with pulsed light of the order of nanoseconds. The subject 102 is a part of a human body or animal, such as a hand, a leg, and a breast. Ultrasonic probes (probes) 103 receive acoustic waves occurring from the subject 102. An internal configuration of the ultrasonic probes 103 will be described below and is therefore omitted here. The ultrasonic probes 103 are made of a structural material such as metal and resin. If a plurality of ultrasonic probes 103 is provided on the acoustic wave unit 100, resin-molded ones are desirably used in consideration of manufacturing cost. The material of the ultrasonic probes 103 may be selected according to that of a support member (hereinafter, referred to as a housing) 104 to be described below. The ultrasonic probes 103 are spirally arranged on the housing 104 to be described below. There are various types of spirals, including a logarithmic spiral and an Archimedean spiral. The present exemplary embodiment uses a spiral with Fibonacci numbers. As illustrated by the arrows in FIG. 2A, when the subject 102 is placed at a measurement position, the ultrasonic probes 103 are directed toward the subject 102 so that their extensions concentrate substantially on one point. The ultrasonic probes 103 are arranged on the housing 104 at substantially the same distances to the subject 102. Such an arrangement of identical ultrasonic probes 103 is effective in terms of cost and replaceability.

The hemispherical housing (hereinafter, referred to as housing) 104 supports the light irradiation unit 101 and the ultrasonic probes 103. Both inside and outside surfaces of the housing 104 are hemispherical in shape. The shape is desirably such that the inside surface having a small radius of curvature and the outside surface having a larger radius of curvature have the same center of curvature. The inside and outside surfaces do not necessarily have the same center of curvature. In the present exemplary embodiment, the housing 104 is hemispherical in shape, whereas the shape of the housing 104 does not necessarily need to be hemispherical as long as the inside surface is concave. The inside surface may have a parabolic, hyperbolic, elliptic, or polygonal shape, or other shapes as long as the reception of acoustic waves is not interfered. Similarly, the outside surface may have a parabolic, hyperbolic, elliptic, or polygonal shape, or other shapes. Since the plurality of ultrasonic probes 103 is fixed to the outside surface, the surface to be fixed of the housing 104 desirably has the same shape. In the present exemplary embodiment, the outside surface is hemispherical. The housing 104 has two types of holes. A first type is a hole for the pulsed light from the light irradiation unit 101 to pass through. Holes of second type are intended for the ultrasonic probes 103 to receive acoustic waves. The housing 104 has a plurality of holes corresponding to the ultrasonic probes 103. The center axes of the latter holes coincide with those of the ultrasonic probes 103.

FIG. 2B is a sectional view illustrating an ultrasonic probe 103 in detail. An acoustic wave conversion element 105 performs mutual conversion between acoustic waves and an electrical signal. In the case of FIG. 2B, the acoustic wave conversion element 105 converts acoustic waves into an electrical signal. Members constituting the acoustic wave conversion element may be made of piezoelectric polymer film material typified by PZT or piezoelectric polymer film material typified by PVDF. The acoustic wave conversion element 105 may be a capacitive element. A CMUT may be used. A sealing member 106 is provided between the housing 104 and the ultrasonic probe 103 so that water and a matching agent, such as a gel, put in the housing 104 to reduce attenuation of the pulsed light will not leak from the housing 104. In FIG. 2B, the sealing member 106 is an O ring. Any member that will not be degraded by water or the matching agent may be used as the sealing member 106. Examples include fluorine-based rubber. Other examples include adhesives. An O ring can be used in consideration of replaceability. In the present exemplary embodiment, one O ring is used, whereas two or more O rings may be provided in consideration of sealability. A circuit 107 processes a signal between the acoustic wave conversion element 105 and a not-illustrated system. In FIG. 2B, the circuit 107 is used for signal processing when a signal is received. The circuit 107 also include a circuit for performing signal processing in reversely transmitting a signal. First wiring 108 connects the acoustic wave conversion element 105 and the circuit 107. Second wiring 109 connects the signal between the circuit 107 and the not-illustrated system. Fixing portions 110 of the ultrasonic probe 103 are portions to be fixed to the housing 104. The fixing portions 110 have a total of two holes through which not-illustrated fastening members are passed. FIG. 2C is a diagram illustrating the shape of the ultrasonic probe 103 in a perspective manner. As illustrated in the diagram, two fixing portions 110 are provided on the outside surface of the ultrasonic probe 103. The fixing portions 110 desirably has a curvature. The fixing portions 110 can be easily fixed to the housing 104 by making the curvature of the fixing portions 110 substantially the same as that along the outside surface of the housing 104.

A method for arranging the ultrasonic probes 103 on the housing 104 will be described in detail. FIG. 2D is a diagram in which some of the ultrasonic probes 103 are displayed on the bottom side of the housing 104. As described above, in the present exemplary embodiment 1, the ultrasonic probes 103 are arranged by using a spiral with Fibonacci numbers. This arrangement is characterized in that adjoining ultrasonic probes 103 are shifted in units of the golden angle (approximately 137.508°) about the axis of rotational symmetry connecting the bottommost portion of the housing 104 and the subject 102. The golden angle is the smaller of the two angles formed by sectioning the circumference according to the golden ratio of 1:(1+√5)/2. The numbers illustrated in FIG. 2D indicate the order of arrangement of the ultrasonic probes 103. The ultrasonic probe 103 at the bottommost portion of the housing 104 is numbered 1. The second ultrasonic probe 103 from the bottommost portion is numbered 2, and the subsequent ones 3, 4, and so on likewise as the distance increases. As described above, the second ultrasonic probe 103 lies at a position shifted from the first ultrasonic probe 103 by 137.508° counterclockwise about the axis of rotational symmetry connecting the bottommost portion of the housing 104 and the subject 102. Similarly, the third ultrasonic probe 103 lies at a position shifted from the second ultrasonic probe 103 by 137.508° counterclockwise about the axis of rotational symmetry connecting the bottommost portion of the housing 104 and the subject 102. The same applies to the subsequent ultrasonic probes 103. As described above, the ultrasonic probes 103 are directed toward the subject 102 so that their extensions concentrate substantially on one point, and are arranged at substantially equal distances to the subject 102 not to interfere with each other. More specifically, this arrangement has characteristics such as represented by the arrows A-A, B-B, and C-C in the diagram. The arrows A-A indicate the 33rd to 135th ultrasonic probes 103. It can be seen that the numbers increase by 34 from the arrow A of the 33rd ultrasonic probe 103 to the arrow A of the 135th ultrasonic probe 103. There is the same relationship as that of the arrows A-A around the axis of rotational symmetry connecting the bottommost portion of the housing 104 and the subject 102. For example, the twelfth ultrasonic probe 103 adjoining the 33rd ultrasonic probe 103 rotationally clockwise has a similar relationship to that of the arrows A-A, i.e., the numbers increase by 34 like 46, 80, 114, and 148. The arrows B-B have a similar concept to that of the arrows A-A. The arrows B-B in the diagram indicate the seventh to 137th ultrasonic probes 103. It can be seen that the numbers increase by 13. Like the arrows A-A, there is the same relationship as that of the arrows B-B around the axis of rotational symmetry connecting the bottommost portion of the housing 104 and the subject 102. The same applies to the arrows C-C. The arrows C-C indicate the seventh to 154th ultrasonic probes 103. Here, the numbers increase by 21. The increments of the arrows A-A, B-B, and C-C, namely, 34, 13, and 21 are all Fibonacci numbers. Although not indicated by arrows, 55 and 89 are also Fibonacci numbers. Take, for example, the seventh ultrasonic probe 103. It can be seen that the numbers of the 62nd and 117th ultrasonic probes 103 increase by 55, and that of the 96th ultrasonic probe 103 by 89. Such an arrangement is maintained from the ultrasonic probe 103 in the bottommost portion of the housing 104 to the farthest ultrasonic probe 103. The ultrasonic probes 103 then need to be spaced at distances to maintain the relationship. For example, in FIG. 2D, the first and 22nd ultrasonic probes 103 are spaced at such a distance, and so the 14th and 35th ultrasonic probes 103. As employed herein, the distance refers to that between the reference points of the ultrasonic probes 103. In FIG. 2D, the distance refers to that between the centers of the circles, i.e., between the intersections of the center axes of the ultrasonic probes 103 and the respective acoustic wave conversion elements 105. The same applies to the other ultrasonic probes 103 since the relationship is maintained as described above. Take the 56th ultrasonic probe 103 instead of the first ultrasonic probe 103. In such a case, the 22nd ultrasonic probe 103 is replaced by the 77th ultrasonic probe 103, the 14th by the 69th, and the 35th by the 90th. Again, such a relationship applies even to the ultrasonic probe 103 farthest from the bottommost portion of the housing 104. The distances between the first, 22nd, 14th, and 35th ultrasonic probes 103 have an unequal relationship, and a quadrilateral is formed by connecting the reference points of the four ultrasonic probes 103. More specifically, the distance between the first and 22nd ultrasonic probes 103 is the shortest. The distance increases between the 14th and 35th ultrasonic probes 103, and then between the first and 14th. The distance between the 22th and 35th is the longest.

FIG. 2E is a detailed view of the state of arrangement of the ultrasonic probes 103 attached to the outside surface of the housing 104. In the diagram, the second wiring 109 is not illustrated. In the diagram, two points obtained by projecting the reference points of adjoining ultrasonic probes 103 on the bottommost side upon an arbitrary plane perpendicular to the axis of rotational symmetry connecting the bottommost portion of the housing 104 and the subject 102 form an angle D with respect to the intersection of the axis of rotational symmetry with the arbitrary perpendicular plane. Similarly, in the diagram, two points obtained by projecting the reference points of adjoining ultrasonic probes 103 having the same relationship, lying at positions away from the bottommost portion, upon the arbitrary plane perpendicular to the axis of rotational symmetry connecting the bottommost portion of the housing 104 and the subject 102 form an angle E with respect to the intersection of the axis of rotational symmetry with the arbitrary perpendicular plane. As illustrated in the diagram, the fixing portions 110 are provided on the outer surfaces of the ultrasonic probes 103. The ultrasonic probes 103 are therefore easy to interfere with each other. As described above, the density of occupation of the ultrasonic probes 103 is substantially equal even in areas farthest from the bottommost portion of the housing 104. It is shown that since the ultrasonic probes 103 are at equal densities in the bottommost portion of the housing 104 and in the farthest areas, the number of ultrasonic probes 103 decreases on the bottom side of the housing 104. The angle D is the largest on the bottommost side of the housing 104. The angle D decreases gradually with an increasing distance from the bottommost portion, and becomes small in the farthest areas. A more detailed description will be given. FIG. 2F is an enlarged view of the arrangement of the ultrasonic probes 103 in the bottommost portion of the housing 104. In the diagram, two points obtained by projecting the reference points of adjoining ultrasonic probes 103 on the bottommost side upon an arbitrary plane perpendicular to the axis of rotational symmetry connecting the bottommost portion of the housing 104 and the subject 102 form an angle F with respect to the intersection of the axis of rotational symmetry with the arbitrary perpendicular plane. FIG. 2G is an enlarged view of the arrangement of the ultrasonic probes 103 in the farthest position from the bottommost portion of the housing 104. In the diagram, like FIG. 2F, two points obtained by projecting the reference points of adjoining ultrasonic probes 103 in the position farthest from the bottommost portion upon an arbitrary plane perpendicular to the axis of rotational symmetry connecting the bottommost portion of the housing 104 and the subject 102 form an angle G with respect to the intersection of the axis of rotational symmetry with the arbitrary perpendicular plane. It can be seen that the angle F in FIG. 2F is greater than the angle G in FIG. 2G. From the foregoing, it is shown that the angle formed between the fixing portions 110 of the ultrasonic probes 103 decreases with an increasing distance from the bottommost side of the housing 104. In other words, on the bottommost side of the housing 104, the fixing portions 110 of the ultrasonic probes 103 are more likely to approach adjacent ultrasonic probes 103.

In view of the foregoing, the ultrasonic probes 103 can be arranged so that the angle F on the bottommost side of the housing 104 becomes small, compared to the angle G. For that purpose, when the ultrasonic probes 103 are arranged as illustrated in FIG. 2D, the distance between the reference points of the first and second ultrasonic probes 103 projected on the axis of rotational symmetry of the housing 104, connecting the bottommost portion of the housing 104 to the subject 102, is the widest. The distance is then reduced with an increasing distance from the bottommost portion. FIG. 2H is a diagram illustrating that the reference points of the first and second ultrasonic probes 103 are projected upon the axis of rotational symmetry of the housing 104, connecting the bottommost portion of the housing 104 to the subject 102. In the diagram, n is the distance between the reference points of the first and second ultrasonic probes 103 projected upon the axis of rotational symmetry of the housing 104, connecting the bottommost portion of the housing 104 to the subject 102. In the diagram, if n is increased, the second ultrasonic probe 103 moves in a direction from the bottommost portion of the housing 104 to the subject 102. By repeating such an increase, the 14th, 22nd, and 35th ultrasonic probes 103 illustrated in the diagram also move. FIG. 21 is a diagram illustrating that the first, 14th, 22nd, and 35th ultrasonic probes 103 are projected upon an arbitrary plane perpendicular to the axis of rotational symmetry connecting the bottommost portion of the housing 104 and the subject 102. In the diagram, dotted lines indicate the ultrasonic probes 103 moved from the conventional positions in the direction from the bottommost portion of the housing 104 to the subject 102. In the diagram, an angle H is formed between lines that connect the reference point of the 14th ultrasonic probe 103 before the movement and the reference point of the first ultrasonic probe 103, projected upon the arbitrary plane, to the intersection of the arbitrary plane with the axis of rotational symmetry. In the diagram, an angle J is formed between lines that connect the reference point of the 14th ultrasonic probe 103 after the movement and the reference point of the first ultrasonic probe 103, projected upon the arbitrary plane, to the intersection of the arbitrary plane with the axis of rotational symmetry. The reference points of the ultrasonic probes 103 are illustrated by bullets at the centers of the circles. From the diagram, it can be seen that the angle H decreases to the angle J before and after the movement of the 14th ultrasonic probe 103. It is easily conceivable that the 22nd and 35th ultrasonic probes 103 also make a similar change accordingly. In other words, the angles corresponding to the angle F described in FIG. 2F become narrower, whereby the interference between the ultrasonic probes 103 can be suppressed.

Such a relationship can be expressed by the mathematical expression described below. Suppose that a distance (hereinafter, referred to as an adjoining distance) between arbitrary reference points of the first and second ultrasonic probes 103 projected upon the axis of rotational symmetry of the housing 104, connecting the bottommost portion of the housing 104 to the subject 102, is n1. Assume n2, n3, . . . ns−1 (s is the total number of ultrasonic probes 103) in succession. Then, the following expression (1) holds:

n1>n2>n3> . . . >ns−1, (1)

where s is the total number of ultrasonic probes 103.

Such a relationship arises from that the angle D in FIG. 2E decreases stepwise to the angle E with an increasing distance from the bottommost portion of the housing 104.

Each of the adjoining distances n may be arbitrarily determined. The adjoining distances n may be determined as appropriate with the ultrasonic probes 103 mounted on the housing 104, whereby the plurality of ultrasonic probes 103 can be arranged at high density.

The angle F described in FIG. 2F decreases with an increasing distance from the bottommost portion. The adjoining distances n may thus safely be made equal from the middle. In such a case, the adjoining distances n can be expressed by the following expression:

n1>n2>n3>n4>n5=n6 . . . =ns−1, (2)

where s is the total number of ultrasonic probes 103.

In expression (2), the adjoining distances n5 and later are made equal. Which position to start making the adjoining distances n equal depends on the shape and size of the ultrasonic probes 103 and the size of the housing 104, and can thus be determined as appropriate based on actual implementation. The total sum of the adjoining distances n in expression (2) is desirably equal to or smaller than the total sum of the adjoining distances n in expression (1). The reason is that if the total sum of the adjoining distances n in expression (2) is greater, the ultrasonic probes 103 are not arranged at density higher than in the arrangement determined by expression (1).

The ultrasonic probes 103 have the shape illustrated in FIG. 2B. However, this is not restrictive. FIG. 2J illustrates an alternative structure of the ultrasonic probes 103. In the diagram, a male screw 111 is formed on the ultrasonic probe 103. The housing 104 is illustrated with hatching. A female screw is similarly formed in the housing 104 to mesh with the male screw 111. As can be seen from the diagram, since the diameter of the male screw 111 is greater than that of the sealing member 106, the hole formed in the housing 104 becomes greater. The ultrasonic probe 103 illustrated in FIG. 2B has the fixing portions 110 formed outside. If a comparison between the shapes of the ultrasonic probes 103 illustrated in FIGS. 2B and 2J shows that the structure illustrated in FIG. 2J can improve the mounting density, the ultrasonic probes 103 can be mounted at higher density by fixing the ultrasonic probes 103 by using the fixing portions provided in the outside surface of the housing 104. FIG. 2K is a diagram illustrating a case in which the structure illustrated in FIG. 2J is employed in FIG. 21. In FIG. 2K, the dotted line of the first ultrasonic probe 103 indicates the size of the ultrasonic probe 103 in FIG. 2B. The solid line represents the ultrasonic probe 103 of FIG. 2J. As described above, the solid line has a diameter that is greater as much as the formation of the male screw 111. As can be seen from the diagram, the four ultrasonic probes 103 illustrated in solid lines are almost next to each other. The ultrasonic probes 103 are provided to satisfy the relationship of expression (1) or (2), whereby the ultrasonic probes 103 can be spaced and arranged as illustrated by the dotted lines. In the present configuration, the ultrasonic probes 103 need to be rotated and inserted when mounted on the housing 104. If high precision positioning is needed, a positioning mechanism needs to be provided aside from the present structure.

FIG. 2L is a diagram for describing a second alternative structure of the ultrasonic probe 103. In the diagram, an abutting portion 112 is provided on the outer periphery of the ultrasonic probe 103. The abutting portion 112 is abutted against an abutting surface provided inside the housing 104. A press ring 113 is intended to press the abutting portion 112 of the ultrasonic probe 103. A male screw is formed on the outer periphery of the press ring 113. A female screw is formed in the housing 104 to mesh with the male screw. In the alternative structure described in FIG. 2J, the ultrasonic probe 103 is rotated when mounted on the housing 104. In the second alternative structure of FIG. 2L, the ultrasonic probe 103 is inserted until the abutting portion 112 is abutted against the housing 104, and the press ring 113 is screwed in for fixing. Although not illustrated in the diagram, if the abutting portion 112 includes an orientation-determining mechanism, such as a D-cut in part of the ultrasonic probe 103, and the housing 104 is machined accordingly, the orientation of the ultrasonic probe 103 can also be determined. Since the present structure is configured so that the ultrasonic probe 103 is pressed and fixed by the press ring 113, the diameter of the press ring 113 is always greater than that of the sealing member 106. However, the present structure may be used if, like FIG. 2J, the structure described in FIG. 2L can improve the mounting density. The relationship is similar to that illustrated in FIG. 2K.

FIGS. 2B, 2J, and 2L illustrate the alternative structures of the ultrasonic probe 103, in which a mechanical fastening method using a screw or a press ring is used as the fixing method. Other fixing methods include one using an adhesive. However, as described above, the housing 104 needs to hold water and the adhesive needs to be reliably removed if the ultrasonic probe 103 fails. In view of replaceability, mechanical fastening methods are therefore desirable.

In FIG. 2F, the ultrasonic probes 103 are directed with the fixing portions 110 vertical with respect to the drawing. However, the ultrasonic probes 103 may be rotated to an arbitrary angle. For example, in the diagram, the ultrasonic probes 103 may be rotated 90° about their axes of rotation. The ultrasonic probes 103 can thus be arranged at high density by directing the ultrasonic probes 103 to optimum angles and selecting the adjoining distances as appropriate.

As described above, the ultrasonic probes 103 can be arranged at high density and an image can be obtained with high reliability by setting the adjoining distances of the ultrasonic probes 103 as expressed by expression (1) or expression (2).

Exemplary embodiment 2 will be described. In exemplary embodiment 1, the reference points of the ultrasonic probes 103 projected upon the axis of rotational symmetry of the housing 104, connecting the bottommost portion of the housing 104 to the subject 102, are described to have a relationship in distance as expressed by expression (1) or expression (2). The ultrasonic probes 103 can thereby be arranged at high density and an image can be obtained with high reliability. Such a method has a high degree of freedom since the adjoining distances are arbitrarily selected according to the structure. However, the method is far from being efficient in terms of design if several hundred or more ultrasonic probes 103 are arranged on the housing 104. The adjoining distances in the direction of the axis of rotational symmetry can desirably be set by using a specific relationship. In view of this, the adjoining distances are expressed by mathematical expression (3):

$$ni = \frac{( A - ( \frac{i}{s - 1} ) )}{{\sum }_{i = 1}^{s - 1} ( A - ( \frac{i}{s - 1} ) )} \times ( Z max - Z min ) \qquad ( 3 )$$

In expression (3), ni is a difference in distance between the reference points of ith and (i+1)th ultrasonic probes 103 projected upon the axis of rotational symmetry of the housing 104, connecting the bottommost portion of the housing 104 to the subject 102. i indicates the ith ultrasonic probe 103 counted from the bottommost portion (the ultrasonic probe 103 at the bottommost portion is the first). A is a constant (hereinafter, referred to as constant A) greater than or equal to 1, and s is the total number of ultrasonic probes 103. Zmax is the position of the reference point of the sth ultrasonic probe 103 projected upon the axis of rotational symmetry of the housing 104, connecting the bottommost portion of the housing 104 to the subject 102. Zmin is the position of the reference point of the first ultrasonic probe 103 projected upon the axis of rotational symmetry of the housing 104, connecting the bottommost portion of the housing 104 to the subject 102. It will be understood that s is an integer greater than or equal to 2 since expression (3) expresses the relationship of arrangement of ultrasonic probes 103.

Expression (3) will be described in more detail. In expression (3), “s−1” expresses the number of distances between the reference points of the ultrasonic probes 103 projected upon the axis of rotational symmetry of the housing 104, connecting the bottommost portion of the housing 104 to the subject 102. Dividing “i” by “s−1” and subtracting the quotient from the constant A calculates a ratio between the ith and (i+1)th ultrasonic probes 103. As i increases, the value of “i” divided by “s−1” increases, and the result of the subtraction of the quotient from the constant A decreases in value. The farther an ultrasonic probe 103 is from the bottommost portion, the smaller the value. It can be seen that if the number i is changed from 1 to s−1 and the results are compared, there holds the relationship of expression (1). The result is then divided by a denominator. The denominator is the total sum of the foregoing results. The division by the denominator normalizes the ratio between the ith and (i+1)th ultrasonic probes 103. For confirmation, the total sum of the first to (s−1)th ratios is calculated, which can be seen to be 1. Finally, the normalized ratio is multiplied by “Zmax−Zmin”. Multiplying the ratio by the difference between the reference points of the ultrasonic probes 103 at the bottommost portion and at the farthest position from the bottommost portion, projected upon the axis of rotational symmetry of the housing 104 connecting the bottommost portion of the housing 104 to the subject 102, calculates the ith height.

The constant A in expression (3) will be further described in detail. FIG. 2M is a diagram for describing a relationship between the constant A and the value of “i” divided by “s−1”. FIGS. 2N and 2O are diagrams illustrating the relationship in specific values for better understanding of FIG. 2M. In both FIGS. 2N and 2O, s is 20. In FIG. 2N, the constant A is 2. In FIG. 2O, the constant A is 5. For convenience of description, i is illustrated to only range from 1 to 3. As described above, the value of “i” divided by “s−1” is always less than or equal to 1. In FIG. 2M, if “i=1”, the value of “i” divided by “s−1” is calculated to be 0.053. The same applies to FIGS. 2N and 2O. Meanwhile, differences α, β, and γ (white framed areas in the diagrams) of the values obtained by dividing “i” by “s−1” from the constant A depend on the constant A. Specifically, if the constant A is small, the value of “i” divided by “s−1” has a high impact on the differences α, β, and γ. If the constant A is large, the value of “i” divided by “s−1” has a relatively low impact. Such a relationship can also be seen from FIGS. 2N and 2O. More specifically, in FIG. 2N, the differences α, β, and γ are calculated to be 1.947, 1.895, and 1.842. The ratio of γ to α is 0.946. In FIG. 2O, the differences α, β, and γ are calculated to be 4.947, 4.895, and 4.842. The ratio of γ to α is 0.979. The greater the constant A, the lower the impact. FIG. 2P is a diagram illustrating an enlarged view of the adjoining distances n1, n2, and n3 calculated by expression (3). As can be seen from expression (3), the adjoining distances n1, n2, and n3 have a proportional relationship with the differences α, β, and γ. The ratios described with reference to FIGS. 2N and 20 determine the heights of the adjoining distances n1, n2, and n3.

Take another example to continue the description. FIG. 2Q illustrates specific calculations of the differences “ni” between the reference points of the ith and (i+1)th ultrasonic probes 103 projected upon the axis of rotational symmetry of the housing 104, connecting the bottommost portion of the housing 104 to the subject 102, when the constant A is 2. FIG. 2R illustrates specific calculations when the constant A is 20. In FIGS. 2Q and 2R, the total number s is 20. For the sake of simplicity, “Zmax−Zmin” is assumed to be 1. In FIG. 2Q, the ratio of n19 to n1 is 0.514. In FIG. 2R, the ratio is 0.953. That is, “ni” can be determined by selecting the constant A. In reality, the constant A is tentatively set and the ultrasonic probes 103 are arranged on the housing 104, and then the constant A can be set to maximize the density. The smaller the constant A, the greater the differences of the adjoining distances. In such a case, the adjoining distances may be too narrow at positions farthest from the bottommost portion of the housing 104. On the other hand, if the constant A is extremely large, the differences of the adjoining distances decrease. In such a case, the ultrasonic probes 103 are arranged at near equal pitches, and the adjoining distances become narrow near the bottommost portion.

In the present exemplary embodiment 2, the relationship of expression (2) described in exemplary embodiment 1 does not hold. The reason is that while the adjoining distances in exemplary embodiment 1 are arbitrarily determined, the adjoining distances in exemplary embodiment 2 are calculated by expression (3). It should be appreciated that the mounting density decreases if the relationship of expression (2) is introduced into expression (3). For example, suppose that the adjoining distances n5 to n19 in FIG. 2R have the same value as that of the adjoining distance n5. In such a case, the total sum of the adjoining distances n1 to n19 exceeds 1, whereas the total sum in FIG. 2R is 1.

As described above, the positions of several hundred or more ultrasonic probes 103 can be efficiently determined by using expression (3) for the adjoining distances of the ultrasonic probes 103.

Note that expression (3) is just an example and not restrictive. It will be understood that examples of similar concepts are also applicable.

Exemplary embodiment 3 will be described. FIG. 2S is a diagram illustrating an acoustic wave unit 300 without the light irradiation unit 101 in FIG. 2A according to exemplary embodiment 1. Similar components to those of exemplary embodiment 1 are designated by the same reference numerals. A description thereof will be omitted.

In FIG. 2S, as described in FIG. 2A, the ultrasonic probes 103 include and use acoustic wave conversion elements 105 for transmission and reception. Specifically, transmission signals are transmitted from a not-illustrated system to the acoustic wave conversion elements 105 and converted into acoustic waves. The converted acoustic waves are emitted to the subject 102 and reach the subject 102. The reached acoustic waves are reflected by the subject 102, converted by the acoustic wave conversion elements 105 in the ultrasonic probes 103 again, and transmitted to the not-illustrated system.

The relationship between the ultrasonic probes 103 and the housing 104 is similar to that in FIG. 2A. As described above, exemplary embodiment 3 can provide similar effects to those described in exemplary embodiments 1 and 2.

Exemplary embodiment 4 will be described. FIG. 2T is a diagram illustrating that the acoustic wave unit 100 illustrated in FIG. 2A according to exemplary embodiment 1 is used and applied to an information acquisition apparatus (subject information acquisition apparatus). FIG. 2T is applicable to both exemplary embodiments 1 and 2. In the diagram, components similar to those of exemplary embodiments 1 and 2 are designated by the same reference numerals. A description thereof will be omitted. The arrows in the diagram represent a flow of pulsed light, acoustic waves, or signals.

A light source 401 emits pulsed light of the order of nanoseconds. The light source 401 can be selected according to the subject 102. For example, if the subject 102 is a living body, a light source 401 having a wavelength of approximately 600 nm to 1500 nm may be selected. The pulsed light emitted from the light source 401 is passed through the light irradiation unit 101 to irradiate the subject 102. The subject 102 absorbs light energy from the irradiated pulsed light and expands instantaneously to generate acoustic waves. The generated acoustic waves are captured by the plurality of ultrasonic probes 103, and signal intensities and phase information are transmitted to a subject information acquisition unit 402. The subject information acquisition unit 402 reconstructs an image based on position information about the ultrasonic probes 103 and the obtained signals. An image display 403 displays the image.

As described above, the subject 102 is irradiated with the pulsed light generated from the light source 401. Elastic waves occurring from the subject 102 are captured by the ultrasonic probes 103, and image reconstruction is performed based on the signals obtained from the ultrasonic probes 103 and the position information about the ultrasonic probes 103, whereby a subject image is obtained.

Exemplary embodiments 3 and 4 differ in the presence or absence of the light irradiation unit 101. Exemplary embodiment 4 is the same as exemplary embodiment 3 in that the received signals are transmitted to the subject information acquisition unit 402 and displayed on the image display 403. Exemplary embodiment 4 thus provides similar effects to those of exemplary embodiment 3.

### Background and Problem of Third Exemplary Embodiment

A third exemplary embodiment will be described. Japanese Patent Application Laid-Open No. 2016-47125 discusses a photoacoustic apparatus including a hemispherical acoustic wave detector on which a plurality of acoustic elements is arranged, and a cup-shaped holding member which holds a region to be inspected of a subject. FIG. 3H is a schematic diagram of the photoacoustic apparatus. FIG. 3H illustrates a cross section of the photoacoustic apparatus. In FIG. 3H, a light irradiation unit 810 irradiates a subject 801 with light. A plurality of reception elements (probes) 813 is built in a hemispherical housing (acoustic wave detector 803) so that the plurality of reception elements 813 is opposed to the subject 801 which is held by a cup-shaped subject holding member 802. FIG. 3H illustrates a state in which the acoustic wave detector 803 moves in a negative direction on an X-axis to irradiate near an end portion 905 of the subject 801 with light. The disclosure discussed in Japanese Patent Application Laid-Open No. 2016-47125 suppresses variations in the contrast of obtained image data by changing the gain of an amplifier in a control unit according to a relative position between the subject 801 and the reception elements 813. This disclosure contributes greatly to the field of diagnostic images.

As illustrated in FIG. 3H, in the photoacoustic apparatus discussed in Japanese Patent Application Laid-Open No. 2016-47125, the plurality of reception elements (probes) 813 is built in the hemispherical housing to be opposed to the subject 801. The other sides of the reception elements 813 are protruded in part from the surface of the other side of the housing. Japanese Patent Application Laid-Open No. 2016-47125 discusses an example of arranging 128 reception elements 813, assuming a breast as the subject 801, whereas no specific method for assembling the plurality of reception elements 813 to the hemispherical housing is discussed. The photoacoustic apparatus discussed in Japanese Patent Application Laid-Open No. 2016-47125 has excellent performance, is relatively expensive, and is not the kind of product to be mass produced. The manufacturing of such a photoacoustic apparatus has not yet been automated by using a robot. To assemble the plurality of probes 813 to a housing employed for such an apparatus that may be regarded as a single product, for example, the plurality of probes 813 including acoustic wave reception elements and the hemispherical housing are prepared, and then the probes 813 are typically assembled to respective predetermined positions of the hemispherical housing in succession by manual operations.

As described above, Japanese Patent Application Laid-Open No. 2016-47125 discusses an example of arranging 128 reception elements 813, assuming a breast as the subject 801. In consideration of increasing the number of reception elements (probes) 813 to increase density for improved resolution, such a conventional assembly method has a problem that probes 813 assembled before become an obstacle and make high density arrangement difficult. The third exemplary embodiment is directed to the provision of an acoustic wave unit in which a plurality of probes can be efficiently arranged on the housing.

According to the third exemplary embodiment, a plurality of probes 813 is efficiently arranged on the housing so that the acoustic wave conversion elements of the probes 813 can be arranged at high density. This improves the detection sensitivity of subject information and enables acquisition of image information with high resolution. Workability during maintenance, such adjustment of the assembled probes 813 and replacement of a failed probe 813, also improves.

### Detailed Description of Third Exemplary Embodiment

Details of the third exemplary embodiment will be described below with reference to the drawings.

Exemplary embodiment 1 will be described. FIGS. 3A-1 and 3A-2 are schematic diagrams illustrating an example of an acoustic wave unit 100 according to the present exemplary embodiment in a schematic manner. FIG. 3A-1 is a sectional view illustrating a cross section of an X-X′ part in FIG. 3A-2. FIG. 3A-2 is a plan view seen in the direction of the arrow Y in FIG. 3A-1. For plain description, a subject 102 is omitted in FIG. 3A-2. In FIGS. 3A-1 and 3A-2, a housing 108 has a concave surface with respect to the subject 102. More specifically, the surface of the housing 108 opposed to the subject 102 has a concave shape with respect to the subject 102, and the housing 108 has a hemispherical shape including the concave portion. A light irradiation unit 101 irradiates the subject 102 with pulsed light of the order of nanoseconds. The light irradiation unit 101 is assembled to an opening 136 formed in the housing 108. A plurality of probes 103a and 103b receives acoustic waves occurring from the subject 102. The plurality of probes 103a and 103b is assembled to a plurality of openings 135 formed in the housing 108. As illustrated in FIGS. 3A-1 and 3A-2, the plurality of probes 103a and 103b is arranged along the concave surface of the housing 108. The probes 103a and 103b receive the acoustic waves from the subject 102 in a plurality of directions, whereby the resolution of the obtained information image can be improved. That is, the acoustic wave unit 100 according to the present exemplary embodiment includes the probes 103a and 103b which receive acoustic waves from the subject 102, and the housing 108 which holds the plurality of probes 103a and 103b arranged thereon. To facilitate the understanding of the characteristics of the present exemplary embodiment, FIGS. 3A-1 and 3A-2 illustrate an example in which a relatively small number of probes 103a and 103b are arranged.

As illustrated in FIGS. 3A-1 and 3A-2, in the present exemplary embodiment, the plurality of probes 103a and 103b is alternately arranged so that different probes 103a and 103b adjoin each other. As can be seen from FIG. 3A-1, the surfaces of the plurality of probes 103a and 103b held by the housing 108, opposed to the subject 102 constitute substantially the same surface.

FIGS. 3B-1 and 3B-2 are sectional views schematically illustrating the probes 103a and 103b. FIG. 3B-1 illustrates the probe 103a. FIG. 3B-2 illustrates the probe 103b.

In FIGS. 3B-1 and 3B-2, an acoustic wave conversion element (also referred to as an electromechanical conversion element or a transducer) 104 is arranged inside a column-shaped cover. The acoustic wave conversion element 104 performs mutual conversion between acoustic waves and an electrical signal. In the present exemplary embodiment, the acoustic wave conversion element 104 converts acoustic waves into an electrical signal. Specifically, the probes 103a and 103b each include the acoustic wave conversion element 104 which receives the acoustic waves from the subject 102 and converts information about the acoustic waves into an electrical signal. The surfaces of the probes 103a and 103b on the side where the acoustic wave conversion element 104 is located are opposed to the subject 102. Members constituting the acoustic wave conversion element 104 may be made of piezoelectric polymer film material typified by PZT or piezoelectric polymer film material typified by PVDF. The acoustic wave conversion element 104 may be a capacitive element. A CMUT may be used. Column-shaped covers 120 in which the acoustic wave conversion elements 104 are arranged may be selected as appropriate in consideration of the shape of vibration parts constituting the acoustic wave conversion element 104. Examples include a rectangular column-shaped cover and a cylindrical cover. In the present exemplary embodiment, for example, the probes 103a and 103b are described to use cylindrical covers as the column-shaped covers 102 in which the acoustic wave conversion elements 104 are arranged. A circuits 106 processes a signal transmitted and received between the acoustic wave conversion element 104 and a not-illustrated system. In the present exemplary embodiment, the circuit 106 is used for signal processing when a signal is received. The circuit 106 may include a circuit for performing signal processing in reversely transmitting a signal. The circuit 106 and the not-illustrated system transmit and receive signals via wiring 107. Mounting portions 110 are used when the probes 103a and 103b are assembled to the housing 108 illustrated in FIGS. 3A-1 and 3A-2. Relative positions of the respective probes 103a and 103b to the housing 108 are determined by the mounting portions 110.

In the present exemplary embodiment, a length Lb from the mounting portions 110 of the probe 103b to the rear end (the side opposite from the acoustic wave conversion element 104) is longer than a length La of the same part of the probe 103a.

In FIGS. 3A-1, 3A-2, 3B-1, and 3B-2, the probes 103a and 103b are inserted into the respective plurality of openings 135 of the housing 108 from the outside surface of the housing 108 (the surface opposite from the surface opposed to the subject 102). The relative positions of the probes 103a and 103b are determined and fixed by using the mounting portions 110. As a result, the probes 103a and 103b protrude from the outside surface of the housing 108 (the surface opposite from the surface opposed to the subject 102) such that the protruding length (Lb) of the probes 103b is longer than the protruding length (La) of the probes 103a. In other words, the probes 103a and 103b are arranged to protrude in part from the surface of the housing 108 on the side opposite from the surface opposed to the subject 102, and the lengths by which the probes 103b and 103b protrude from the surface on the opposite side differ between the plurality of probes 103a and 103b. In consideration of replacement and maintenance of the probes 103a and 103b, the probes 103a and 103b are desirably fixed by manual operations using fastening members such as screws and connectors, not by adhesion.

A procedure for assembling the probes 103a and 103b will be described. The plurality of probes 103a having a small protruding length (La) from the outside surface of the housing 108 is initially assembled. The probes 103b having a large protruding length (Lb) are then assembled. That is, the probes 103a and 103b can be attached and detached from the outside surface of the housing 108. The plurality of probes 103a and 103b are divided into a plurality of groups, and the protruding lengths differ depending on the groups. In such a configuration, the protruding portions of the probes 103a assembled before do not become an obstacle in assembling the probes 103b. The distances between the probes 103a and 103b can therefore be reduced for high density mounting, which enables acquisition of image information with high resolution. Another effect is that during maintenance such as adjustment and replacement of the assembled probes 103a and 103b, the probes 103b having a large protruding length (Lb) can be easily operated since the surrounding probes 103a each have short protruding portions (La) and do not become an obstacle.

Another effect is that the probes 103a with a small protruding length (La) can also be easily operated by temporarily detaching some of adjacent probes 103b, as compared to when probes of the same lengths are arranged.

In consideration of the efficiency of assembly and replacement of the probes 103a and 103b by manual operations and the size of the acoustic wave unit 100, the difference between the lengths of the different probes 103a and 103b (difference between La and Lb) is typically suitably set in the range of 3 mm to 50 mm. The difference can be desirably set in the range of 5 mm to 40 mm, and optimally in the range of 7 mm to 35 mm.

The diameter of the cylindrical covers, which is equivalent to the diameter of the probes 103a and 103b, is determined as appropriate in consideration of the size and shape of the vibration parts, which affect the characteristic (frequency band) of the acoustic wave conversion element 104, and the number of probes 103a and 103b arranged. The diameter is typically suitably set in the range of 5 mm to 15 mm. The diameter can be desirably set in the range of 6 mm to 12 mm, and optimally in the range of 7 mm to 10 mm.

Exemplary embodiment 2 will be described. FIG. 3C is a plan view schematically illustrating another exemplary embodiment of the acoustic wave unit 100 according to the third exemplary embodiment. FIG. 3C is a plan view corresponding to FIG. 3A-2 of exemplary embodiment 1.

Exemplary embodiment 2 is similar to exemplary embodiment 1 except for the arrangement of the probes 103a and 103b on the housing 108. That is, in the acoustic wave unit 100 of the present exemplary embodiment, the housing 108 also has a hemispherical shape including a concave portion. In FIG. 3C, like exemplary embodiment 1, the light irradiation unit 101, and the plurality of probes 103a having a small protruding length and the plurality of probes 103b having a large protruding length from the outside surface of the housing 108 are each assembled to the housing 108. In exemplary embodiment 2, the probes 103a and 103b are arranged in a plurality of spiral configurations for higher mounting density. The probes 103a having a small protruding length and the probes 103b having a large protruding length are alternately arranged along the spirals. More probes 103a having a small protruding length are provided near the center of the housing 108 (near the light irradiation unit 101). Such an arrangement enables mounting at even higher density without impairing workability during assembly and maintenance. In the present exemplary embodiment, the probes are divided into two groups, with two types of protruding lengths corresponding to the respective groups. In view of workability during assembly and maintenance, the probes may be divided into three or more types. In FIG. 3C, a distance D between two probes 103b having a long protruding length is assumed in a direction substantially orthogonal to a spiral direction A along which the probes 103a and 103b are arranged.

The direction D is determined as appropriate in consideration of the diameter of the probes 103a and 103b and the number of arranged probes 103a and 103b. In view of efficiency during assembly and replacement of the probes 103a and 103b by manual operations, the distance D is typically suitably set in the range of 3 mm to 30 mm. The distance D can be desirably set in the range of 5 mm to 20 mm, and more desirably in the range of 7 to 15 mm.

The target subject 102 of the present exemplary embodiment may be a breast, a hand, or a foot of a living body, other regions of a living body, or a non-living substance. For example, assuming an apparatus that can measure a breast as the subject 102, the hemispherical housing 108 including the concave portion may have a hemispherical radius (inner radius) in the range of 100 mm to 150 mm. The hemispherical radius (inner radius) can be desirably set in the range of 110 mm to 130 mm. In a specific example of the present exemplary embodiment, an acoustic wave unit 100 may be configured so that 900 to 1100 probes 103a and 103b having a cylindrical cover diameter in the range of 7 mm to 10 mm are arranged on a housing 108 having a hemispherical radius (inner radius) in the range of 110 mm to 130 mm.

Exemplary embodiment 3 will be described. FIG. 3D is a plan view schematically illustrating another exemplary embodiment of the acoustic wave unit 100 according to the third exemplary embodiment.

In exemplary embodiment 3, some of the probes 103a and 103b spirally arranged in exemplary embodiment 2 are replaced with probes 103c having a protruding length different from those of the probes 103a and 103b. The probes 103c having the different protruding length are arranged as guideposts according to a rule (such as every 10 probes), so that the individual probes can be easily identified even if the mounting density and number of probes 103a, 103b, and 103c are increased.

Exemplary embodiment 4 will be described. FIGS. 3E-1 and 3E-2 are schematic diagrams illustrating an acoustic wave unit 200 without the light irradiation unit 101 of FIGS. 3A-1 and 3A-2 according to exemplary embodiment 1 in a schematic manner. FIG. 3E-1 is a sectional view illustrating a cross section of an X-X′ part in FIG. 3E-2. FIG. 3E-2 is a plan view seen in the direction of the arrow Y in FIG. 3E-1. For plain description, a subject 102 is omitted in FIG. 3E-2. In exemplary embodiment 4, a probe 103a is also attached to the position of the light irradiation unit 101.

Like exemplary embodiment 1, the probes 103a and 103b include respective acoustic wave conversion elements 104, and perform transmission and reception by using the acoustic wave conversion elements 104. Specifically, transmission signals are transmitted from a not-illustrated system to the acoustic wave conversion elements 104, and converted into acoustic waves. The converted acoustic waves are emitted to the subject 102 and reach the subject 102. The reached acoustic waves are reflected by the subject 102, and converted into electrical signals again by the acoustic wave conversion elements 104 in the probes 103a and 103b. The electrical signals are transmitted to the not-illustrated system. The relationship between the probes 103a and 103b and the housing 108 is similar to that in exemplary embodiment 1. Similar effects to those described in exemplary embodiment 1 can thus be provided.

Exemplary embodiment 5 will be described. FIGS. 3F-1 and 3F-2 are schematic diagrams illustrating an acoustic wave unit 300 of flat type in a schematic manner. FIG. 3F-1 is a sectional view illustrating a cross section of an X-X′ part in FIG. 3F-2. FIG. 3F-2 is a plan view seen in the direction of the arrow Y in FIG. 3F-1. For plain description, a subject 102 is omitted in FIG. 3F-2. A housing 109 has a flat surface with respect to the subject 102. Like exemplary embodiment 4, a plurality of probes 103a and 103b performs transmission and reception with respect to the subject 102. The plurality of probes 103a and 103b is assembled to a plurality of openings 137 formed in the housing 109. As illustrated in FIGS. 3F-1 and 3F-2, the probes 103a having a small protruding length and the probes 103b having a large protruding length from the outside surface of the housing 109 (surface opposite from the surface opposed to the subject 102) are arranged in an alternate pattern. Such an arrangement enables high density mounting without impairing workability during assembly and maintenance.

Exemplary embodiment 6 will be described. FIG. 3G is a diagram illustrating that the acoustic wave unit 100 according to exemplary embodiment 1 is used and applied to a subject information acquisition apparatus. While a part of exemplary embodiment 1 is used in the diagram, other exemplary embodiments may be used. In the diagram, similar components to those of exemplary embodiment 1 are designated by the same reference numerals. A description thereof will be omitted.

A light source 401 emits pulsed light of the order of nanoseconds. The light source 401 can be selected according to the subject 102. For example, if the subject 102 is a living body, a light source 401 having a light wavelength of approximately 600 nm to 1500 nm may be selected. The pulsed light emitted from the light source (light source unit) 401 is passed through the light irradiation unit 101 to irradiate the subject 102. The subject 102 absorbs light energy from the irradiated pulsed light and expands instantaneously to generate acoustic or ultrasonic waves. The generated acoustic or ultrasonic waves are captured by the plurality of probes 103a and 103b, and signal intensities and phase information are transmitted to a subject information acquisition apparatus 402. The subject information acquisition apparatus (signal processing unit) 402 reconstructs an image based on position information about the probes 103a and 103b and the obtained signals. An image display 403 displays the image.

As described above, the subject 102 is irradiated with the pulsed light generated from the light source 401. Elastic waves (acoustic waves) occurring from the subject 102 are captured by the probes 103a and 103b. Image reconstruction is performed based on the signals obtained from the probes 103a and 103b and the position information about the probes 103a and 103b, whereby the information about the subject 102 can be reconstructed as an image. If the acoustic wave units 200 and 300 according to exemplary embodiments 4 and 5 are used, there is a difference in that the light irradiation unit 101 is absent, but the received signals are similarly transmitted to the subject information acquisition apparatus 402 and then displayed on the image display 403.

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2016-225553, filed Nov. 18, 2016, No. 2016-254366, filed Dec. 27, 2016, and No. 2016-254367, filed Dec. 27, 2016, which are hereby incorporated by reference herein in their entirety.

What is claimed is:

1. A probe array comprising:
a cup-shaped support member having a plurality of through holes; and
a probe provided in a through hole of the plurality of through holes, the probe including a transducer configured to be capable of conversion between an acoustic wave and an electrical signal,
wherein the probe include a fixing portion for fixing the probe to an outer surface of the support member, the fixing portion having a curvature.

2. The probe array according to claim 1, wherein a surface of the fixing portion has a curvature substantially the same as that of a surface of the outer surface.

3. The probe array according to claim 1, wherein the transducer is provided so that a reception surface of the transducer is directed toward an inside of an inner surface of the support member.

4. The probe array according to claim 1, wherein the transducer is provided so that normals to reception surfaces of the transducers of the respective probes intersect at one point.

5. The probe array according to claim 1, wherein the transducer is a capacitive transducer.

6. The probe array according to claim 1, wherein the transducer is a piezoelectric transducer.

7. A probe array comprising:
a cup-shaped support member having a plurality of holes; and
a cylindrical probe provided in a hole of the plurality of holes, the cylindrical probe including a transducer configured to be capable of conversion between an acoustic wave and an electrical signal,
wherein an axis of rotational symmetry of the cup-shaped support member and normals from points at which a curved surface including an inside surface of the cup-shaped support member intersects with center axes of the cylindrical probe to the axis of rotational symmetry have a plurality of intersections, and
wherein a distance between a first intersection among the plurality of intersections and a second intersection adjoining the first intersection on an apex side of the cup-shaped support member is greater than a distance between the first intersection and a third intersection adjoining the first intersection on a side opposite from the apex side.

8. The probe array according to claim 7, wherein a distance between adjoining intersections among the plurality of intersections decreases in a direction away from the apex on the axis of rotational symmetry.

9. The probe array according to claim 7, wherein the plurality of intersections being numbered from 1 to s, s being a total number of probes, in order in a direction away from the apex on the axis of rotational symmetry, a distance ni between an ith intersection (1<i<s; s is a positive integer) and an (i+1)th intersection satisfies expression (3):
$$ni = \frac{( A - ( \frac{i}{s - 1} ) )}{{\sum }_{i = 1}^{s - 1} ( A - ( \frac{i}{s - 1} ) )} \times ( Z max - Z min ) \qquad ( 3 )$$
where A is a constant greater than or equal to 1, Zmin is a distance from the apex to an intersection located closest to the apex among the plurality of intersections, and Zmax is a distance from the apex to an intersection located farthest from the apex among the plurality of intersections.

10. The probe array according to claim 7, wherein the probe includes a capacitive transducer.

11. The probe array according to claim 7, wherein the probe includes a piezoelectric transducer.

12. An acoustic wave unit comprising:
a housing configured to hold a plurality of probes arranged thereon, each probe of the plurality of probes including an acoustic wave conversion element configured to receive an acoustic wave from a subject and convert information about the acoustic wave into an electrical signal, each probe of the plurality of probes being provided to protrude in part from a surface of the housing on a side opposite from a surface opposed to the subject,
wherein each probe of the plurality of probes has a protruding length from the surface on the opposite side that is different from another probe of the plurality of probes.

13. The acoustic wave unit according to claim 12, wherein the plurality of probes is divided into a plurality of groups, and the protruding length of each probe of one group of the plurality of groups is different from that of each probe of another group of the plurality of groups.

14. The acoustic wave unit according to claim 12, wherein the surface of the housing opposed to the subject has a concave shape with respect to the subject.

15. The acoustic wave unit according to claim 14, wherein the housing has a hemispherical shape including a concave portion.

16. The acoustic wave unit according to claim 15, wherein surfaces of the plurality of probes held by the housing, the surfaces being opposed to the subject, constitute substantially the same surface.

17. The acoustic wave unit according to claim 14, wherein surfaces of the plurality of probes on a side where the acoustic wave conversion elements are located are opposed to the subject.

18. The acoustic wave unit according to claim 14,
wherein the plurality of probes comprises a first type of probe and a second type of probe,
wherein the first type of probe has a protruding length that is different from a protruding length of the second type of probe, and
wherein the first type of probe and the second type of probe are alternately arranged on the housing along a spiral.

19. The acoustic wave unit according to claim 14, wherein the acoustic wave conversion elements are capacitive acoustic wave conversion elements.
